(19) United States
(12) Patent Application Publication
(10) Pub. No.: US 2020/0010964 A1
(43) Pub. Date: Jan. 9, 2020

(54) METHODS OF AND SYSTEMS FOR ELECTROCHEMICAL REDUCTION OF SUBSTRATES

(71) Applicants: Yusif Abdullayev, Khirdalan (AZ); Zafar Hasanov, Baku (AZ)

(72) Inventors: Yusif Abdullayev, Khirdalan (AZ); Zafar Hasanov, Baku (AZ)

(21) Appl. No.: 16/224,503

(22) Filed: Dec. 18, 2018

Related U.S. Application Data
(60) Provisional application No. 62/694,865, filed on Jul. 6, 2018.

(51) Int. Cl. C25B 3/04; B01D 53/32; C07C 53/06; C07C 53/10; C25B 11/12; C25B 11/03

(52) U.S. Cl. CPC C25B 3/04; B01D 53/326; C25B 11/035; C07C 53/10; C25B 11/12; C07C 53/06

(57) ABSTRACT

Provided are methods and systems for electrochemical reduction of carbon sources including, for example, carbon dioxide and carbonates. The methods and systems use a catalyst. The catalyst may comprise metals such as Fe (iron), and Ti (titanium), Ni (nickel), and Zn (zinc) and/or oxides thereof. The metals may be disposed in an aluminosilicate. The catalyst may be a porous volcanic tuff based material. The methods and systems can be used to produce various carbon-source, reduction products.

### CROSS REFERENCE TO RELATED APPLICATIONS

This application claims priority to U.S. Provisional Application No. 62/694,865, filed on Jul. 6, 2018, the disclosure of which is hereby incorporated by reference.

### FIELD OF THE DISCLOSURE

The disclosure generally relates to catalytic materials for electrochemical reduction of substrates. More particularly the disclosure generally relates to methods and systems for electrochemical reduction of carbon substrates, such as, for example, carbon dioxide and graphite, using catalytic materials.

### BACKGROUND OF THE DISCLOSURE

The capture and utilization of CO₂ addresses anthropogenic climate change while simultaneously providing fundamentally new routes to petrochemical feedstocks, making it a key challenge to the chemistry community. To date, there have been many efforts to define schemes for the capture and conversion of CO₂ to fine chemicals. Electrochemical carbon capture is a promising emerging technology for decreasing greenhouse gas concentrations: Snyder et al. developed the resin-water electrodeionization system to capture and recover CO₂ from flue gases. Electrochemical splitting technology of CaCO₃ was proposed to capture and store CO₂ from the air or a waste system by keeping pH within a certain range. A solid organic-pigment thin film, combining the advantages of electrochemistry with those of an organic solid active material, was used to capture CO₂. It was proposed as an effective and energy saving alternative to using organic bases (amine species) for this purpose. A Pt/K-βAl₂O₃ tubular electrochemical system was designed to capture CO₂ and convert it to potassium carbonates and bicarbonates. Huang and coworkers designed ceramic-carbonate dual-phase membrane application to electrochemically separate CO₂ from a simulated natural gas, and the CO₂ permeation parameters were studied in detail. They also reported a silver-carbonate electrochemical membrane for post-combustion CO₂ capture. Nitrogen-doped porous carbons from waste biomass have been calcinated for realizing the atom economy. In this study, N-doped porous carbons have been successfully prepared from waste tobaccos by a simple pre-treatment process. The sample was calcinated at a high temperature in order to get micro/meso-porous structure for application in electrochemical CO₂ capture. The liquid natural gas thermal plant flue gas (CO₂ concentration: 10%) was captured electrochemically and recovered via vacuum. CO₂ was bubbled through electrolyte solutions (KOH, KHCO₃, KCl, etc.), and its concentration increased due to increased partial pressure.

CO₂ capture and conversion is more desirable than capture-only because it is not limited to the storage of CO₂ in its initial form or as a carbonate. In such processes, CO₂ is converted to valuable organic products currently accessed from petrochemical processing. The following literature broadly discusses CO₂ capture and conversion processes. An electrochemical parallel plate reactor with an ionic liquid was proposed as means to convert CO₂ to methanol at low temperatures and atmospheric pressures. Microalgaes were used to convert CO₂ to biodiesel in an air-lift-type photobioreactor cathode chamber. A critical review of the electrochemical CO₂ conversion to methanol has concluded that 6,7-dimethyl-4-hydroxy-2-mercaptopteridine as a biomimetic catalyst did not catalyze the reaction. The authors suggested a careful re-evaluation of these kind of papers based on GC and NMR results. CO₂ was converted via the titanium (cathode) and platinum (anode) electrochemical system to carbon and oxygen using Li₂O—LiCl molten electrolyte. A similar LiCl—Li₂CO₃ molten electrolyte-supported electrochemical system was used to convert CO₂ to carbon, and it was suggested as a negative electrode material for Li-ion batteries. Patterned Ni electrodes on single crystal electrolyte was used to observe electrochemical behavior of a solid oxide electrochemical cell and solid oxide fuel cell in CO/CO₂ atmosphere. In a more recent review, transition metal (Cu, Cu₂O, Ga₂O₃, Au, Ag, Sn, Bi, Ru, etc.) catalyzed electrochemical conversion of CO₂ to methane, ethylene, methanol, and formic acid was broadly summarized. Iron porphyrins were utilized as electrochemical catalysts to reduce CO₂ to CO, and mechanistic studies were performed to understand electrochemical charge transfer in various acidic media. Electrochemical conversion of CO₂ to formic acid has been achieved by means of Pd-polyaniline/carbon nanotube hybrids. Electrodeposited Cu₂O on a carbon electrode has been used in an electrolysis system for converting CO₂ to mainly ethylene. CO₂ conversion to higher carbon chain (C2-C4) organic products with 10% Faradaic efficiency was achieved by using Cl-induced bi-phasic Cu2-Cu catalyst. CO₂ reduction to CO was studied based on the Au₂₅ electro-catalyst. The solar cell supported optimized electrocatalytic system turnover numbers and efficiency have been determined. Various transition metals (Cu, Co, Fe, and Pt) have been deposited on carbon nanotubes or carbon black and utilized as electrochemical catalysts for converting CO₂ to isopropyl alcohol and acetone. In the presence of a Ni catalyst, N-heterocyclic carbene supported conversion of CO₂ to methane has been conducted. In this review, the heterogeneous catalytic conversion mechanism of CO₂ to organic chemicals has been reported extensively, including some mechanistic theoretical calculations. Multi-walled carbon nanotube surfaces were functionalized with a polymerized ionic liquid (in order to increase CO₂ affinity), and were used as a cathode catalyst support to convert CO₂ to formic acid. Electrochemical conversion of CO₂ into methanol including reaction media, electrochemical cell design, cathode materials, and working condition have been described in this recent review. CO₂ reduction to methanol and methane has been studied on seven (Au, Ag, Zn, Cu, Ni, Pt, and Fe) transition metal surfaces. The product formation depends on binding energy of CO on the catalysts surfaces. They found that only methanol can be produced by means of Au catalyst, whereas methane is the only product of the Fe catalyst. Remaining five metals (Ag, Zn, Cu, Ne, and Pt) can be used in electrochemical system to convert CO₂ to both products. Hexagonal Zn (h-Zn) was utilized as catalyst to convert CO₂ to CO electrochemically. The catalyst material operated for maximum 30 h and it was accepted as the best result. Cu was impregnated on K/Al₂O₃ and utilized to reduce CO₂ to CO and CH₄. Porosity and addition of K into Cu/Al₂O₃ increases CO₂ conversion.

Based on the foregoing, there exists an ongoing and unmet need in the art for improved methods and systems for electrochemical reduction of CO₂.

### SUMMARY OF THE DISCLOSURE

The present disclosure provides methods for electrochemical reduction of substrates such as, for example, carbon dioxide and graphite. The present disclosure also provides systems for electrochemical reduction such as, for example, carbon dioxide and graphite.

The electrochemical methods and/or systems described herein can be used to address global climate change issues through the conversion of CO₂ and/or carbonates into, for example, organic chemical feedstocks previously accessed from petrochemicals. Examples of product streams include, but are not limited to, methanol, ethanol, ethylene oxide, formaldehyde, etc. Variants of tuff from the consolidation of volcanic ash were tested as a stable material for electrocatalysis. The XRD spectra of variants are given in FIGS. 5-10. The green mineral (1a, clinoptilolite-quartz containing tuff) is a desirable electrocatalyst for the conversion of CO₂ into various organic compounds.

In an aspect, the present disclosure provides methods of electrochemical reduction. In an example, a substrate, which may be a carbon source, is reduced. The methods and systems are based on the use of a catalyst (e.g., tuff modifications) in the methods and systems. In an example, naturally available volcanic, porous tuff materials are used as catalysts to convert CO₂ into various organic substances. This material is naturally doped with metals (see, e.g., XRD and SEM spectra provided herein for the tuff content of examples of catalyst materials), and is efficient at reducing CO₂, for example, when placed under a negative bias.

In various examples, a method for electrochemical reduction (e.g., of reducible material such as, for example, a carbon source, hydrogen sulfide, cyanides (e.g., —CN containing materials), nitrates, phosphates, sulfates, and the like) comprises: a) contacting one or more substrate, which may be a carbon source (e.g., carbon dioxide, carbonate, graphite, or cyanide, or a combination thereof), hydrogen sulfide, a nitrate, a phosphate, a sulfate, or a combination thereof, and, optionally, a chloride source such as, for example, a chloride salt (e.g., sodium chloride), with a catalyst of the present disclosure (e.g., a catalyst Fe (iron), and Ti (titanium), Ni (nickel), Zn (zinc) and, optionally, Ga (gallium), disposed in an aluminosilicate) where the catalyst is under an electrochemical potential (e.g., a negative bias), such that reduction products (e.g., carbon source reduction products such as, for example, carbon dioxide reduction products and/or carbonate reduction products) are formed. In an example, a catalyst comprises one or more volcanic tuff modifications. Suitable catalyst materials (e.g., minerals such as, for examples, natural ores) can be found in Azerbaijan. The natural ore is a porous clinoptilolite mineral comprising quartz with silicates of alkaline and alkaline earth metals. The catalyst (e.g., natural ore) can comprise, for example, one or more metals that can act cooperatively or individually as active sites.

In an aspect, the present disclosure provides systems for electrochemical reduction. The systems comprise catalyst materials (e.g., volcanic tuff modifications such as, for example, clinoptiloltite-quartz materials). A system can carry out a method of the present disclosure. The system is an electrochemical system for reducing, for example, carbonates into organic substances. This system can also be used to reduce and convert cyanide (—CN), nitrates, phosphates, sulfates, and the like, into amines, phosphines, sulfides (mercaptanes), respectively. For example, a system contains a batch reactor or a continuous flow reactor with a unique electrode design and catalytic component that receives electricity from DC power supply.

The system comprises an anode and a cathode. The anode and cathode may be in the same or different compartments. One or more electrode (e.g., cathode, anode, working electrode, or a combination thereof) may comprise the catalyst material (e.g., tuff mineral). The catalyst material may be both an electrode and a catalytic material. The system may comprise a conventional two-compartment electrolysis system. The system may be a 2-electrode or 3-electrode system. The system may comprise a bulk electrolysis system. The system may comprise an H-cell design.

The system can reduce sulfate into H₂S (hydrogen sulfide). The system is also capable of incorporating the chloride ion from NaCl solutions into organic substances, with evidence of the formation of chloroform and CCl₄, as identified by GC/FID.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a fuller understanding of the nature and objects of the disclosure, reference should be made to the following detailed description taken in conjunction with the accompanying figures.

FIG. 1 shows and example of an electrochemical batch reactor. Gas outlet (1); lit for feeding the reactor (2); gas collection compartment (3); cooling water outlet (4); semi-cone shaped graphite (5); reactor body (6); mineral (clinoptilolite-quartz) (7); cooling water inlet (8); and DC power supply (9).

FIG. 2 shows tuff contains mainly clinoptilolite and quartz (1a). (Black) type: 2Th/Th locked-Start: 4.875*-Step: 0.020*-Step time: 38.4 s-Temp.: 25° C. (room) -Time started: 0 s-2-Thet Operations: Displacement 0.156|Background 8.128, 1.0001 Import. (Red) 01-085-0794 (A)-Quartz-SiO₂ Y: 42.70%-d x by: 1.-WL:1.54184-Hexagonal-a 4.91000-b 4.91000-c 5.40000-alpha 90.000-beta 90.000-gamma 120.000-Primitive-P3221 (154)-3-1. (Blue) 01-089-7538 (C)-Clinoptilolite-(Na 1.32K1.28Ca1.72Mg0.52)(Al6.77Si29.23072)(H2O)26.84-Y: 35.94%-d x by: 1.-WL: 1.54184-Monoclinic-a 17.07500-b 17.95720-c 7.40910-alpha 90.000. (Green) 01-080-2467 (C)-Sodium Aluminum Silicate Gallium (Na((A10.7Ga0.3)Si308)-Y: 7.40%-d x by: 1.-WL: 1.54184-Triclinic-a 8.17300-b 12.87400-c 7.13000-alpha 93.860-beta 116.480-ga. (Magenta) 01-089-6217 (C)-Potassium Sodium Calcium Aluminum Iron Magnesium Titanium Silicate-(K0.776Na0.181Ca0.011)(A10.916Fe0.016Mg0.011)2(Si0.787Al0.223Ti-Y:12.55%-d x by: 1. WL: 1.54.

FIG. 3 shows an example of tuff that comprises dickite, quartz, and hematite (2). (Black) Locked Coupled 0 Start: 5.001*-End: 80.004*-Step: 0.020*-Step time: 19.2 s-Temp.: 25° C. (Room)-Time Started: 0 s-2-Theta: 5.001*-Theta: 2.501**-Chi: 0.00*-Phi: 0.00*-Operations: Y Scale Add 20|Y Scale Add 20|Strip kAlpha2 0.500|Background 1.000, 1.000|Import. (Red) 01-076-0632 (C)-Dickite 0 Al2Si2O5(OH)4-Y: 23.50%-d x by: 1.-WL: 1.5406-Monoclinic-a 5.15000-b 8.93000-c 14.42400-alpha 90.000-beta 96.730-gamma 90.000-Base-centered-Cc(9)-4-658.782- [symbol] c PDF 1.1-S-Q. (Blue) 01-087-2096 (C)-Quartz low-alpha-SiO2-Y: 21.83%-d x by: 1.-WL: 1.5406-Hexogonal a 4.91270-b 4.91270-c 5.40450-alpha 90.000-beta 90.000-gamma 120.000-Primitive-P3221 (154)-3-112.961- [symbol] c PDF 2.9-S-Q. (Green) 01-089-2810 (C)-Gematite-alpha-Fe2O3-Y: 7.00%-d x by: 1.-WL: 1.5406-Rhombo H axes-a 5.04000-b 5.04000-c 13.75000-alpha 90.000-beta 90.000-gamma 120.000-Primitive-R-3c (167)-6-302.478-Inc PDF 3.-S.

FIG. 4 shows an example of tuff that comprises quartz, kaolinite, and hematite (3). (Black) Type: Locked Coupled-Start: 5.001*-End: 80.004*-Step: 0.020*-Step time: 19.2 s-Temp.: 25° C. (Room)-Time Started: 0 s-2-Theta: 5.001*0 Theta: 2.501*-Chi: 0.00*-Phi: 0.00*-Operations: Y Scale Add 20|Y Scale Add 30|Strip kAlpha2 0.500|Background 1.000, 1.000|Import. (Blue) 01-087-2096 (C)-Quartz low-alpha-SiO2-Y: 21:83%-d x by: 1.-WL: 1.5406-Hexogonal-a 4.91270-b 4.91270-c 5.40450-alpha 90.000-beta 90.000-gamma 120.000-Primitive-P3221 (154)-3-112.961- [symbol] c PDF 2.9-S-Q. (Red) 01-080-0663 (C)-Kaolinite Al2(Si2O5)(OH)4-Y: 17.21%-d x by: 1.-WL: 1.5406-Triclinic-a 5.15770-b 8.94170-c 7.39670-alpha 91.672-beta 104.860-gamma 89.898-Primitive-P1(1)-2-329.571- [symbol] c PDF 1.1-S-Q 6. (Green) 01-072-0469 (C)-Hematite-alpha-Fe2O3-Y: 1.77%-d x by: 1.-WL: 1.5406-Rhombo H axes-a 5.03800-b 5.03800-c 13.77200-alpha 90.000-beta 90.000-gamma 120.000-Primitive R-3c (167)-6-302.722- [symbol] c PDF 3.2.

FIG. 5 shows an example of tuff that comprises quartz, pyrophyllite, kaolinite, and hematite (4). Type: Locked Coupled-Start: 5:001*-End: 80.004*-Step: 0.020*-Step time: 19.2 s-Temp.: 25° C. (Room)-Time Started: 0 s-2-Theta: 5.001*-Theta: 2.501*-Chi: 0.00*0 Phi: 0.00*-Operations: Y Scale Add 20|Y Scale Add 20|Strip kAlpha2 0.500|Background 1.000, 1.000|Import. (Blue) 01-085-0796 (A)-Quartz-SiO2-Y: 22.49%-d x by: 1.-WL: 1.5406-Hexagonal-a 4.91180-b 4.91180-c 5.40340-alpha 90.000-beta 90.000-gamma 120.000-Primiative-P3221 (154)-3-112.896-He PDF 18.6-F29-1000(0. (Red) 00-046-1306 (I)-Pyrophyllite-Al2Si4O10(OH)2-Y: 14.49%-d x by: 1.-WL: 1.5406-Monoclinic-a 5.17500-b 8.90200-c 18.67300-alpha 90.000-beta 100.100-gamma 90.000-Base-centered-C2/c (15)-4-846.894-F3O. (Green) 01-074-1784 (C)-Kaolinite 1A-Al2Si2O5(OH)4-Y: 10.76%-d x by 1.-WL: 1.5406-Monoclinic-a 5. 7500-b 8.90200-c 18.67300-alpha 91.800-beta 104.500-gamma 90.000-Base-centered-C1 (0)-2-327.337- [symbol] e PDF 1.1-F. (Magenta) 01-089-8103 (C)-Hematite-syn-Fe2O3-Y: 6.06%-d. by. 1.-WL: 1.5406-Rhombo H axes-a 5.02060-b 5.02060-c 13.71960-alpha 90.000-beta 90.000-gamma 120.000-Primitive-R-3c (167)-6-299.491- [symbol] e PDF 3.3-F.

FIG. 6 shows an example of tuff that comprises pyrophyllite and hematite (5). (Black) Type: Locked Coupled-Start: 5.001*-End: 80:004*-Step: 0.020*-Step time: 19.2 s-Temp.: 25° C. (Room)-Time Started: 0 s-2-Theta: 5.001*-Theta: 2.501*-Chi: 0.00*-Phi: 0.00*-Operations: Y Scale Add 20|Y Scale Add 20|Strip kAlpha2 0.500|Background 1.000, 1.000|Import. (Red) 00-025-0022 (I)-Pyrophyllite-1A-Al2Si4O10(OH)2-Y: 26.18%-d x by: 1.-WL: 1.5406-Triclinic-a 5.16100-b 8.95700-c 9.35100-alpha 91.030-beta 100.370-gamma 89.750-Base-centered-C-1 (0)-2-425.140-F30-27(0. (Green) 01-085-0599 (A)-Hematite-Fe2O3- Y: 8.70%-d by. 1.-WL: 1.5406- Rhombo H axes-a 5.03811- b 5.03811-c 13.75559-alpha 90.000-beta 90.000-gamma 120.000-Primitive-R-3c (167)-6-302.375- [symbol] e PDF 2.9-F21-1.

FIG. 7 shows an example of tuff that comprises pyrophyllite, kaolinite, and hematite (6). (Black) Type: Locked Coupled-Start: 5.001*-End: 80.004*-Step: 0.020*-Step time: 19.2 s-Temp.: 25° C. (Room)-Time Started: 0 s-2-Theta: 5.001*-Theta: 2.501*-Chi: 0.00*-Phi: 0.00*-Operations: Y Scale Add 20|Y Scale Add 30|Strip kAlpha2 0.50|Background 1.000, 1.000|Import. (Red) 00-046-1308 (I)-Pyrophyllite-2M-Al2Si4O10(OH)2-Y: 20.64% d x by: 1.-WL: 1.5406-Monolinic-a 5.17500-b 8.90200-c 18.67300-alpha 90.000-beta 100.103-gamma 90.000-Base-centered-C2/2 (15)-4-846.894-F30. (Blue) 01-080-0885 (C)-Kaolinite-Al2(Si205)(OH)4-Y: 12.30%-d x by: 1.-WL: 1.5406-Triclinic-a 5.15550-b 8.94380-c 7.40510-alpha 91.700-beta 104.840-gamma 89.800-base-centered-C1 (0)-2-329.909- [symbol] e PDF 1.-F. (Green) 01-086-2368 (A)-Hematite-syn-alpha-Fe2O3-Y: 5.40-d. by. 1.-WL: 1.5406-Rhombo H axes-a.503550-b 5.03550-c 13.74710-alpha 90.000-beta 90.000-gamma 120.000-Primitive-R-3c (167)-6-301.875- [symbol] e PDF.

FIG. 8 shows a cyclic voltammogram in a 10% aqueous carbonate solution using a green tuff as a working electrode. An onset of cathodic current is observed at −0.3 V vs Ag/AgCl, followed by further reduction at −0.75 V vs Ag/AgCl at a scan rate of 500 mV/s.

FIG. 9 shows SEM EDX analysis of different modification of the green mineral (1b).

FIG. 10 shows an example of characterization of a catalyst of the present disclosure.

FIG. 11 shows an example of characterization of a catalyst of the present disclosure.

FIG. 12 shows an example of characterization of a catalyst of the present disclosure.

FIG. 13 shows an example of characterization of a catalyst of the present disclosure.

FIG. 14 shows an example of characterization of a catalyst of the present disclosure.

FIG. 15 shows an example of characterization of a catalyst of the present disclosure.

FIG. 16 shows an example of characterization of a catalyst of the present disclosure.

FIG. 17 shows an example of characterization of a catalyst of the present disclosure.

FIG. 18 shows an example of characterization of a catalyst of the present disclosure.

FIG. 19 shows an example of characterization of a catalyst of the present disclosure.

FIG. 20 shows an example of characterization of a catalyst of the present disclosure.

FIG. 21 shows an example of characterization of a catalyst of the present disclosure.

FIG. 22 shows an example of characterization of a catalyst of the present disclosure.

FIG. 23 shows ICP/MS characterization data for various samples from Table 1.

FIG. 24 shows ICP/MS characterization data for various samples from Table 2.

FIG. 25 shows ICP/MS characterization data for various samples from Table 3.

FIG. 26 shows ICP/MS characterization data for various samples from Table 4.

FIG. 27 shows CO₂ adsorption isotherm related to amount of absorbed CO₂ (at 273K and 298K) vs absolute pressure for sample 1a.

FIG. 28 shows heat (kJ/mol) of CO₂ adsorption vs quantity adsorbed (mmol/g) for sample 1a.

FIG. 29 shows adsorption isosteres of CO₂ (chemisorptive interaction) on the mineral sample (1a).

FIG. 30 shows Brunauer-Emmett-Teller (BET) surface area analysis dataf or quantity (cm³/g) of adsorbed CO₂ vs relative pressure (p/po).

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

When weight percent (weight %) or parts per million (ppm) is used herein to describe the amount of a particular catalyst component, the stated weight percent value refers to the weight percent of the particular catalyst component based on the total weight of the catalyst.

The present disclosure provides methods for electrochemical reduction of substrates. The present disclosure also provides systems for electrochemical reduction of substrates.

The electrochemical methods and/or systems described herein can be used to address global climate change issues, for example, through the conversion of CO₂ and/or carbonates into, for example, organic chemical feedstocks previously accessed from petrochemicals. Examples of product streams include, but are not limited to, methanol, ethanol, ethylene oxide, formaldehyde, etc.

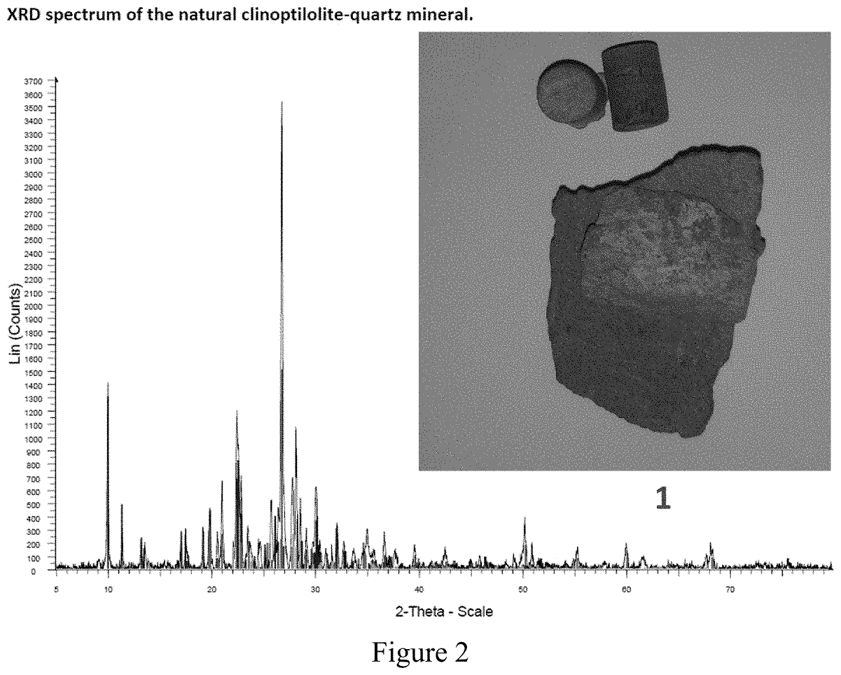

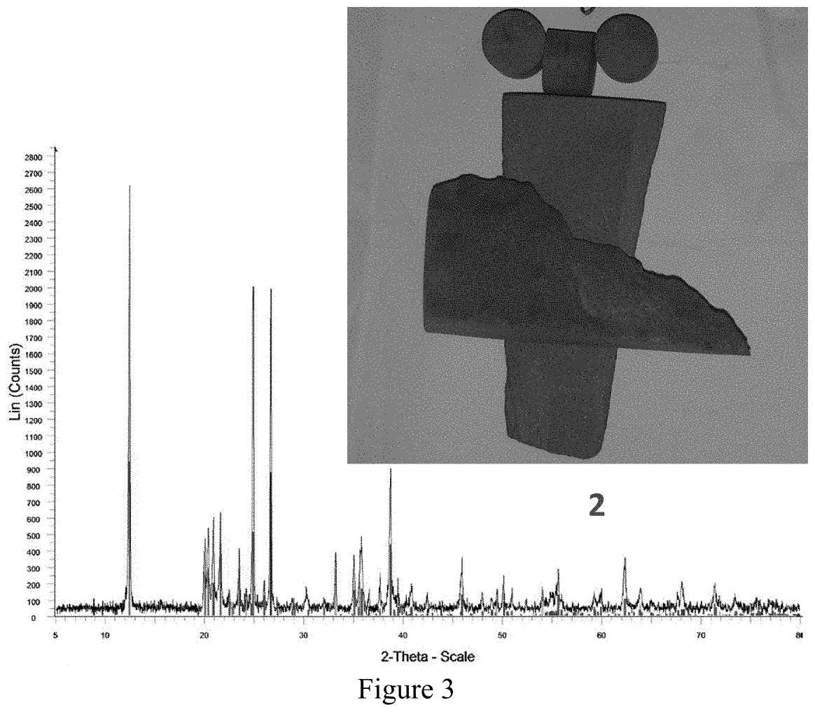

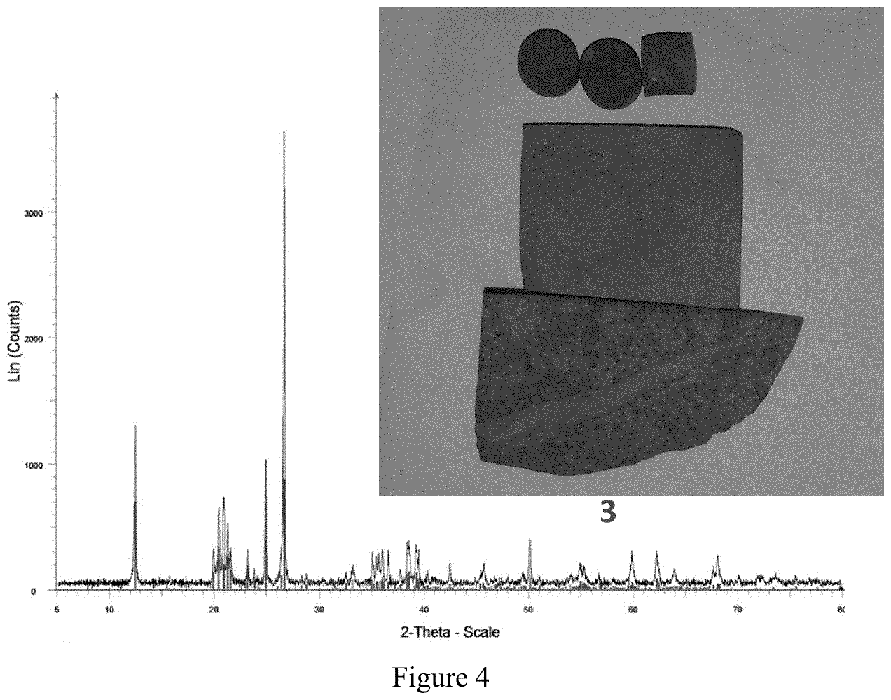

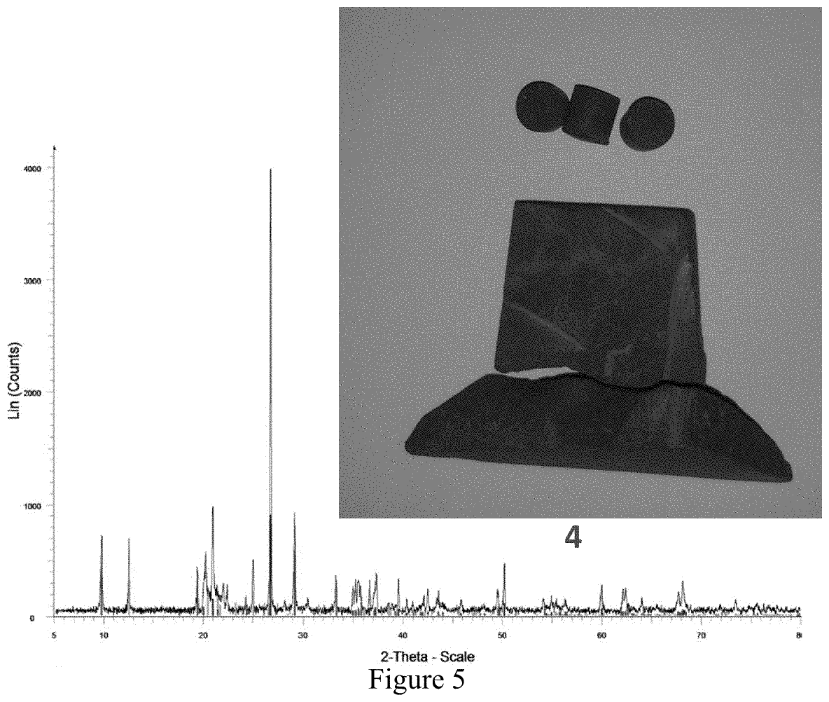

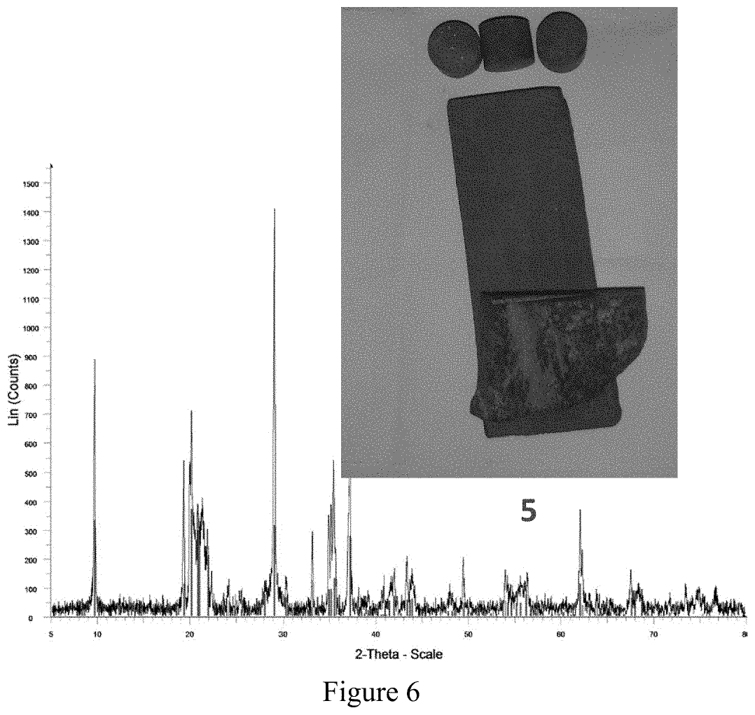

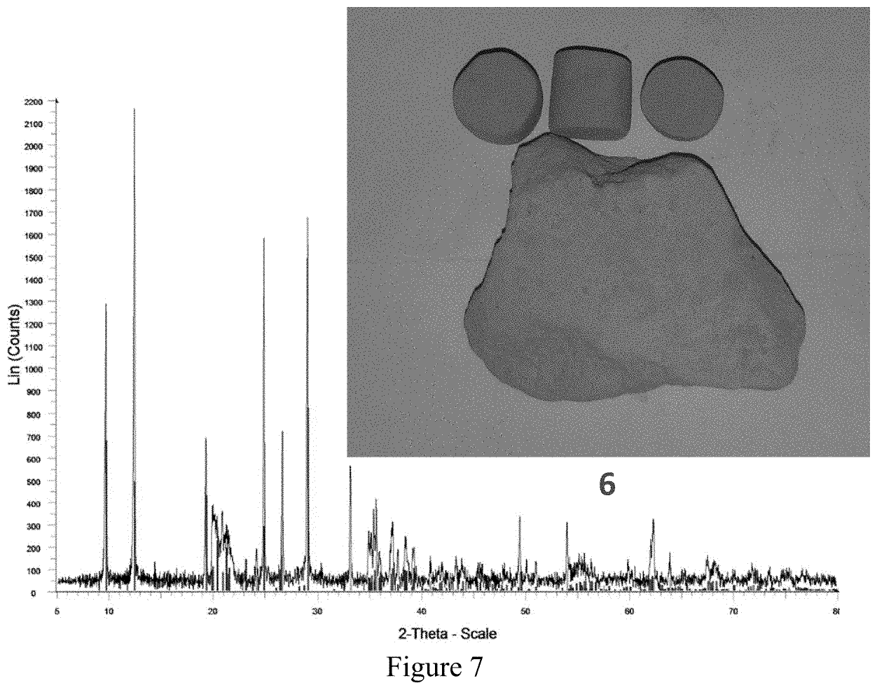

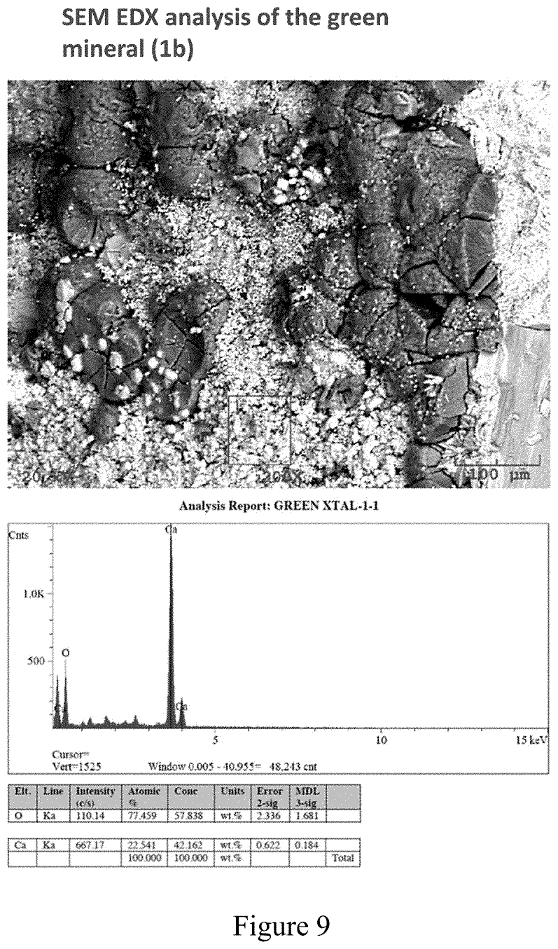

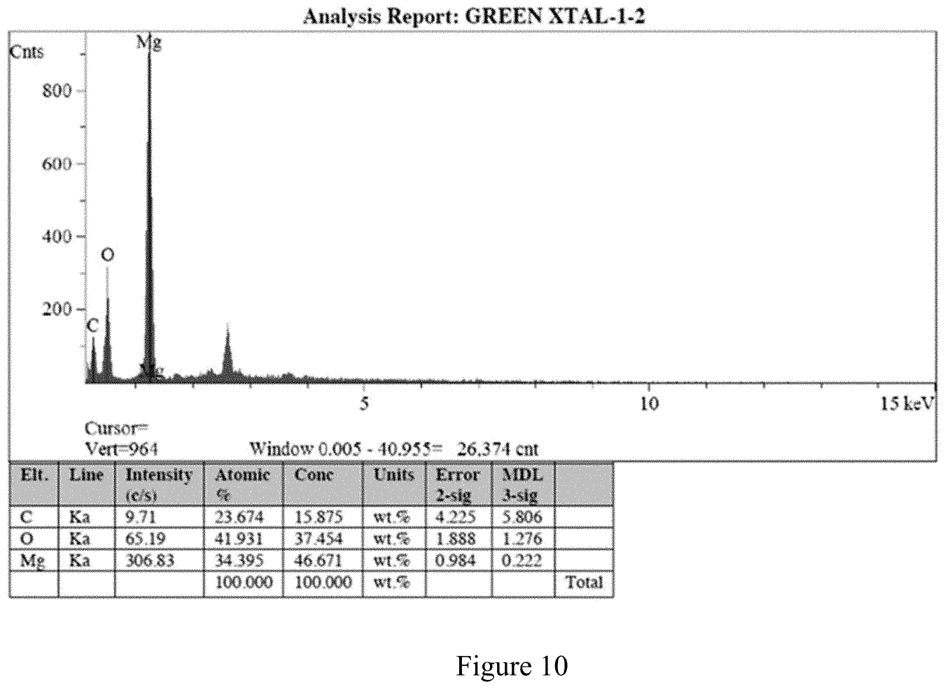

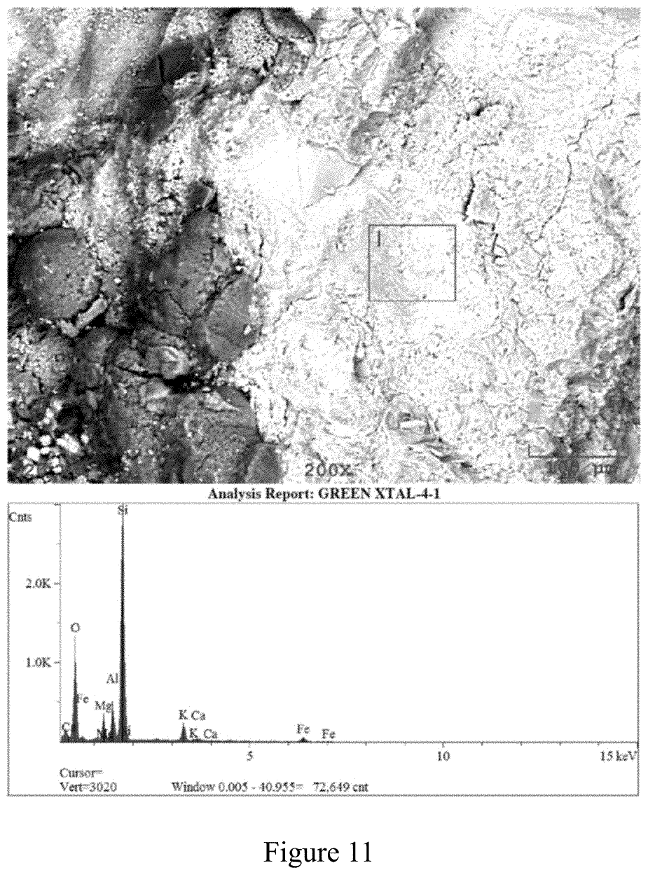

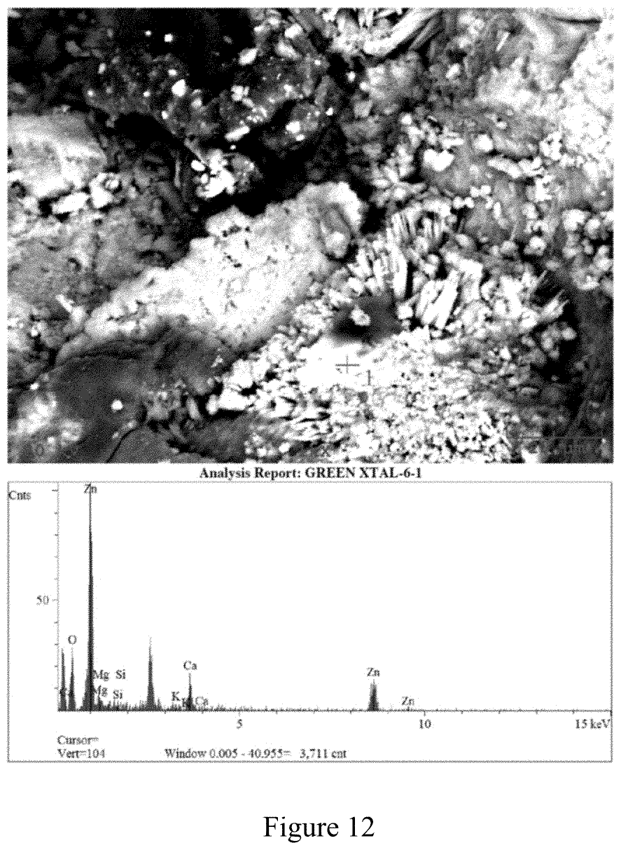

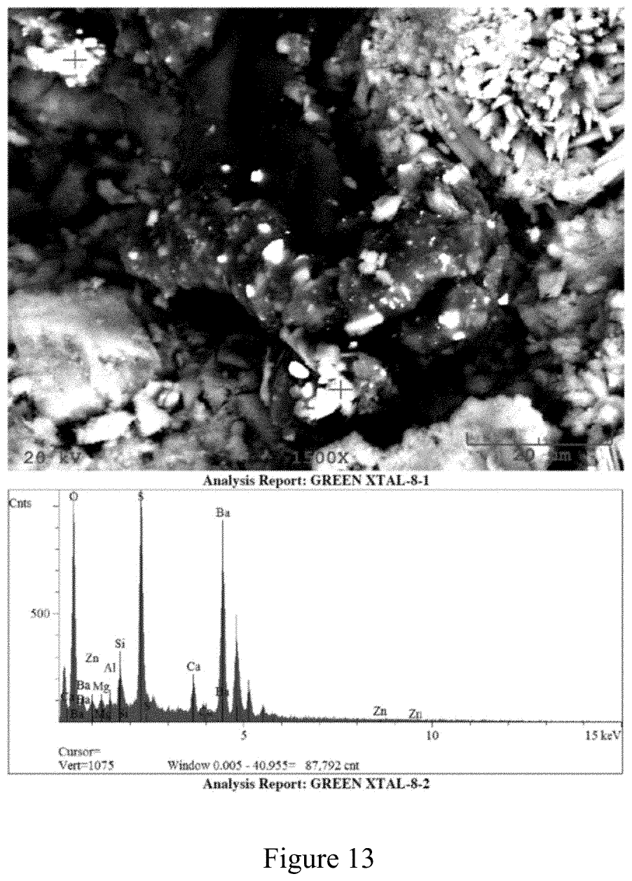

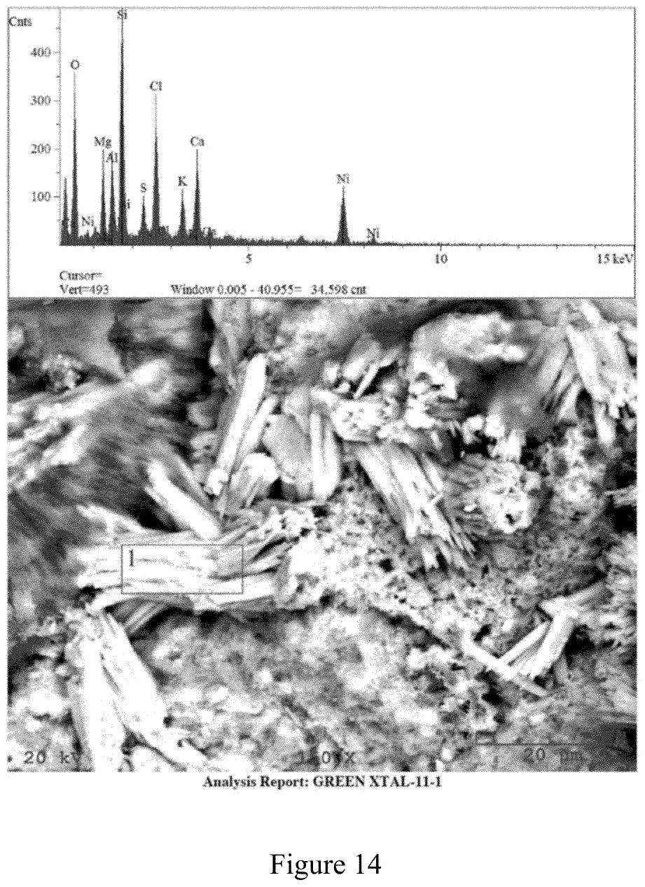

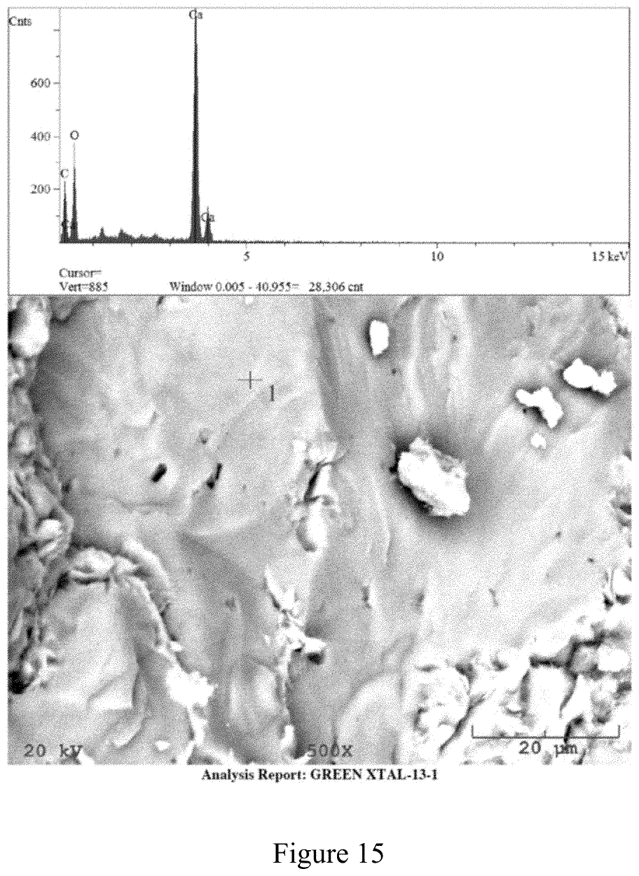

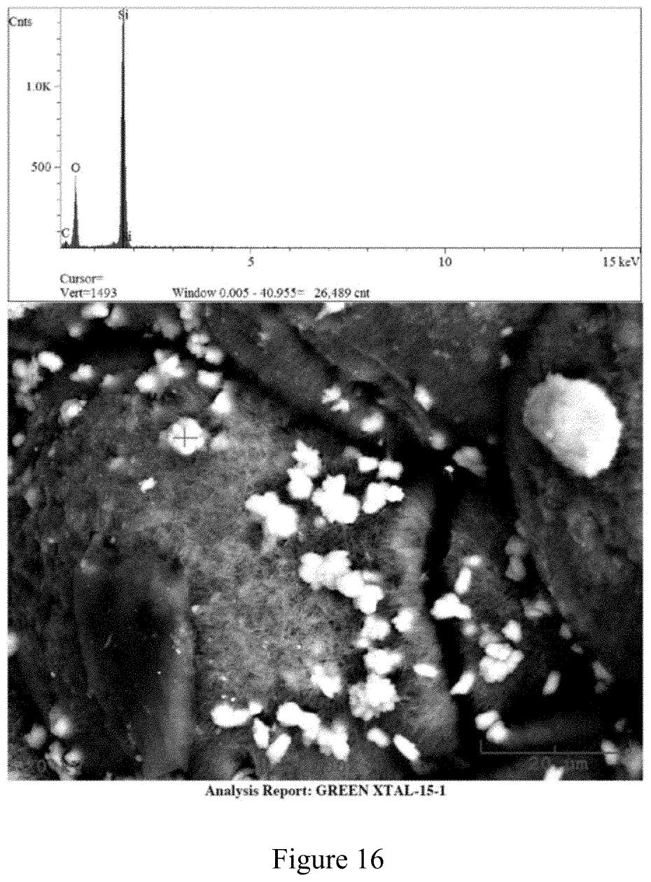

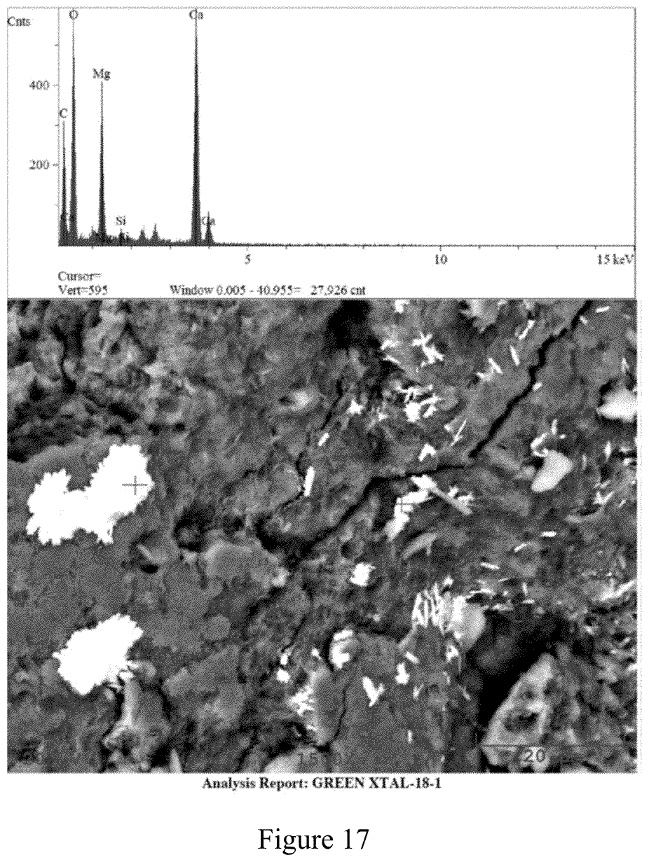

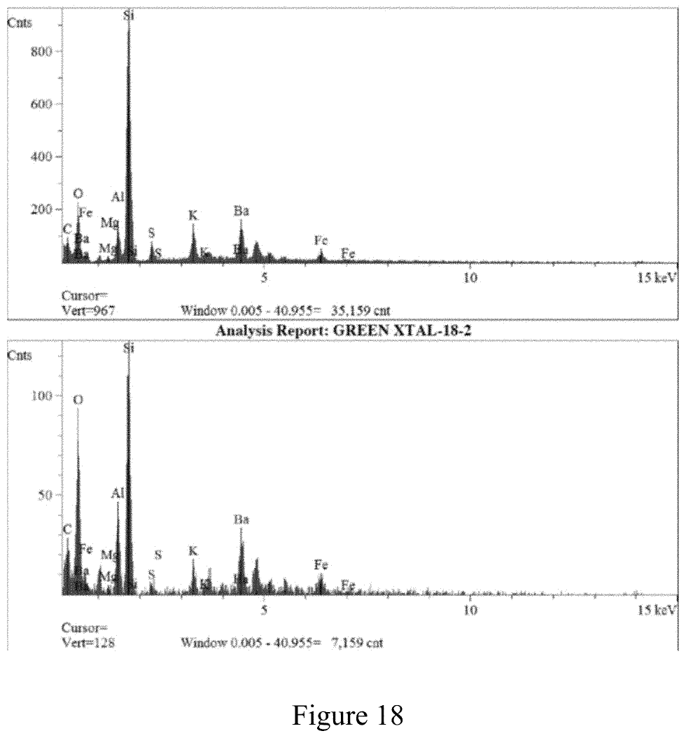

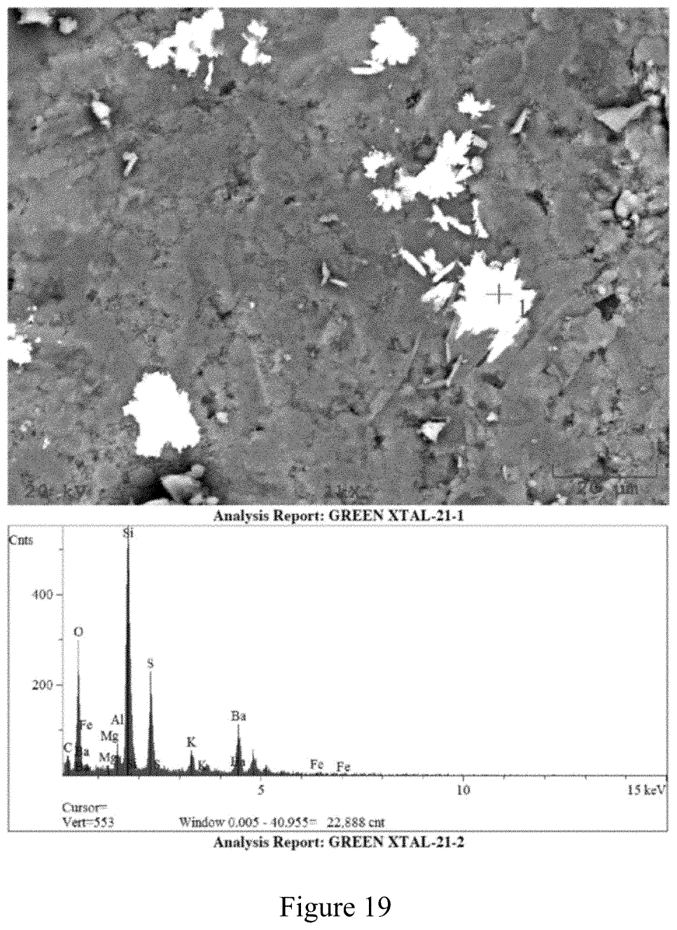

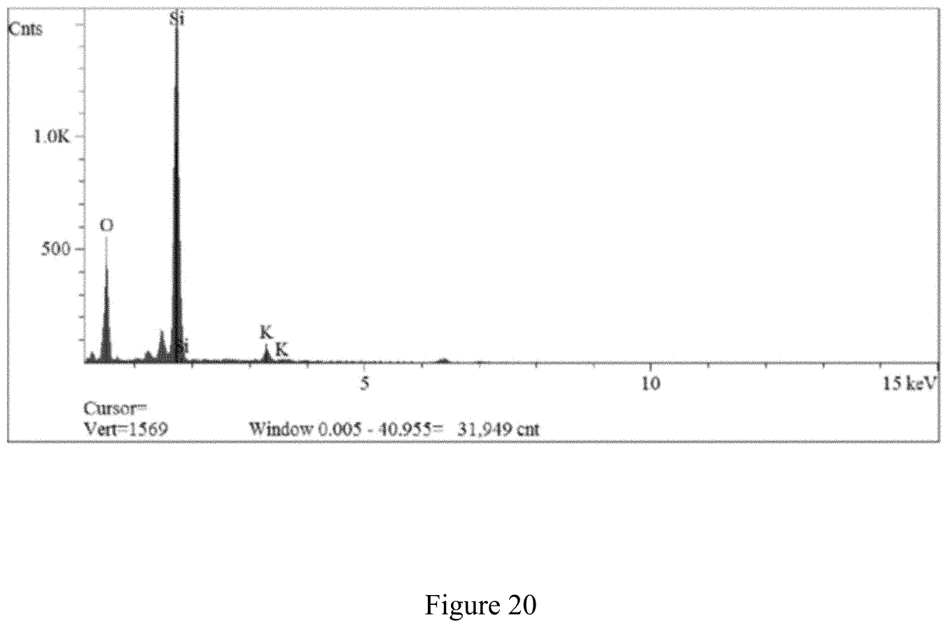

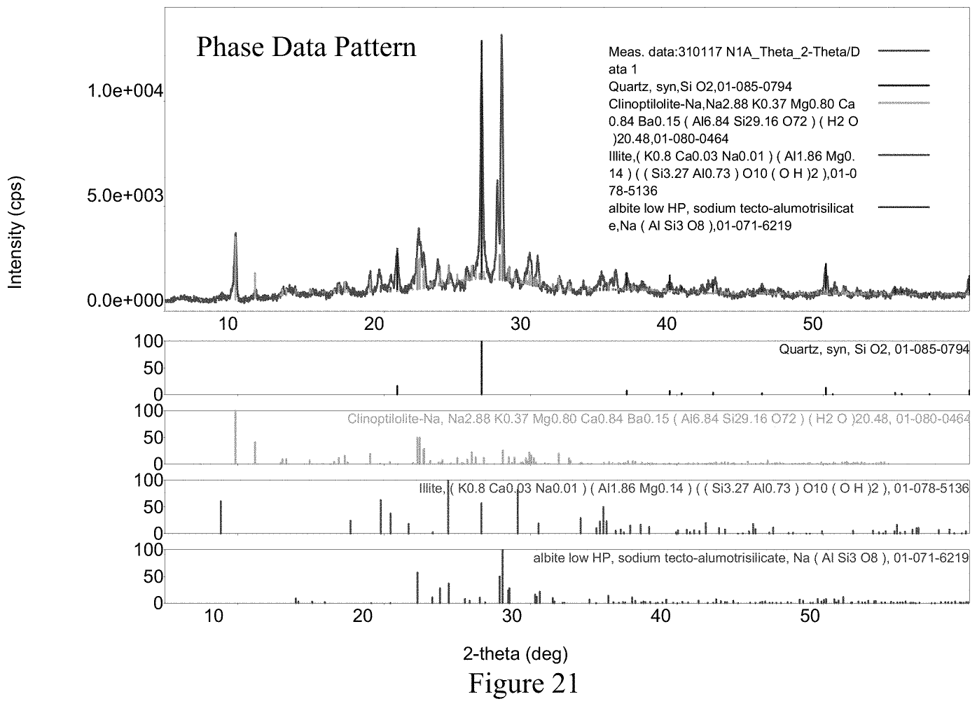

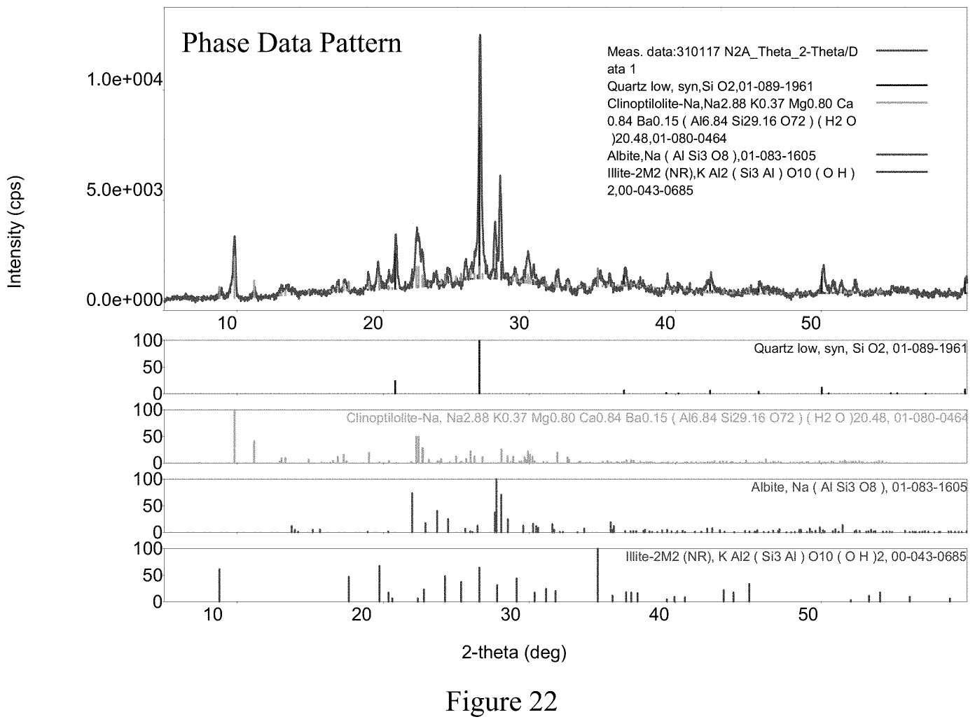

Variants of tuff from the consolidation of volcanic ash were tested as a stable material for electrocatalysis. The XRD spectra of examples of variants are given in FIGS. 2-7. The green mineral (1a, clinoptilolite-quartz containing tuff) is a desirable electrocatalyst, for example, for the conversion of CO₂ into various organic compounds. Optimization experiments show that the stability and catalytic abilities of the minerals decrease from variant 1 to 6, correlated to quartz content. As seen from the XRD spectra, the last two samples (5,6) do not contain quartz. The green mineral (1) is heterogeneous with multiple active domains. SEM EDX analyses of different regions (1b) are given in FIG. 9. This family comprises, in various examples, catalytically active metals such as, e.g., Zn, Ni, Fe, Ga, Ti and etc. in their framework (see FIG. 2 for 1a). In addition to their catalytic activities, the composition of the green variant includes alkali and alkaline earth metals (e.g., Na, K, Ca, Mg, Ba, and the like) Al is the main element of the framework. The stability of the minerals enables operation at high potentials (>10 V). The minerals with gray, green, and brown colors are highly stable and operate for at least couple of months, even under high applied overpotentials. As the minerals are abundant natural resources, this system represents an economically viable alternative to precious metals and metal nanoclusters which are applied as a catalyst with low TOF, and short operating hours in current CO₂ conversion methods. In various examples, the present methods use of CO₂ as a C1 source for organic molecules in scalable schemes. Systems described herein provide novel routes to convert waste stream chemicals to value-added products using electrochemical techniques, representing a green and efficient strategy that will potentially disrupt existing petrochemical-based pathways.

In an aspect, the present disclosure provides methods of electrochemical reduction. In an example, a substrate (e.g., a carbon source) is electrochemically reduced. The methods and systems are based on the use of a catalyst (e.g., tuff modifications) in the methods and systems. In an example, naturally available volcanic, porous tuff materials are used as catalysts to convert substrates (e.g., CO₂ or graphite) into various organic substances. This material is naturally doped with metals (see, e.g., XRD and SEM spectra provided herein for the tuff content of examples of catalyst materials), and is efficient at reducing CO₂, for example, when placed under a negative bias.

In various examples, a method for electrochemical reduction (e.g., of reducible material such as, for example, a carbon source, hydrogen sulfide, cyanides (e.g., —CN containing materials), nitrates, phosphates, sulfates, and the like) comprises: a) contacting one or more substrate, which may be a carbon source (e.g., carbon dioxide, carbonate, graphite, or cyanide, or a combination thereof), hydrogen sulfide, a nitrate, a phosphate, a sulfate, or a combination thereof, and, optionally, a chloride source such as, for example, a chloride salt (e.g., sodium chloride), with a catalyst of the present disclosure (e.g., a catalyst comprising Fe (iron), Ti (titanium), Ni (nickel), Zn (zinc), and, optionally, Ga (gallium) disposed in an aluminosilicate), where the catalyst is under an electrochemical potential (e.g., a negative bias), such that reduction products (e.g., carbon source reduction products such as, for example, carbon dioxide reduction products and/or carbonate reduction products) are formed.

Various substrates can be used. The substrate is a reducible substrate. In an example, a substrate is a carbon source. Other non-limiting examples of substrates include, nitrates, phosphates, sulfates, which may be reduced/converted into, for example, amines, phosphines, sulfides (mercaptans), respectively.

Various carbon sources can be used. For example, CO₂ sources, carbonate sources, carbon sources such as, for example, graphite, cyanide (—CN), or combinations thereof are utilized as a carbon source. Examples of CO₂ sources include, but are not limited to, atmospheric CO₂, compressed CO₂ tanks, and dry ice. Examples of carbonate sources include, but are not limited to, natural carbonate sources, and carbonate salts (potassium, sodium). An electrode may comprise a carbon source.

The substrate may be present as in an aqueous medium or an ionic liquid. The aqueous medium or ionic liquid can be an electrolyte in an electrochemical reaction or reactor. In various examples, an electrolyte in an electrochemical reaction comprises one or more reducible materials. In an example, the aqueous medium is an aqueous sodium chloride solution and the carbon source reduction products comprise chlorinated products (e.g., chlorinated hydrocarbon compounds).

The catalyst may be composite materials or hybrid materials that comprise a catalyst material. For example, a catalyst material (e.g., a clinoptiloltite-quartz mineral) is mechanically incorporated into materials such as, for example, resins, polymers (e.g., porous polymers), gels, layered sheets, pressed powders, or thin films.

In an example, a catalyst comprises one or more volcanic tuff modifications. Suitable catalyst materials (e.g., minerals such as, for examples, natural ores) can be found in Azerbaijan. The natural ore is a porous clinoptilolite mineral comprising quartz with silicates of alkaline and alkaline earth metals. The metals may be present in the catalyst as metal oxides (e.g., one or more naturally-occurring oxide of a metal) and/or metal ions (e.g., metal ions in one or more naturally-occurring oxidation state of a metal). The catalyst (e.g., natural ore) can comprise, for example, one or more metals that can act cooperatively or individually as active sites. Examples of metals and/or metal oxides, include, but are not limited to, Ga (gallium), Fe (iron), Ti (titanium), Zn (zinc), Ni (nickel), Zirconium (Zr), Cupper (Cu), Vanadium (V), and combinations thereof. In an example, the catalyst (e.g., natural ore) comprises three metals (such as, for example, the combination of Ga (gallium), Fe (iron), and Ti (titanium)) that can act cooperatively or individually as active sites. Artificial catalyst designs based on the natural catalyst include, but are not limited to, embedding the catalytically active metals (e.g., Ga (gallium), Fe (iron), Ti (titanium), Zn (zinc), and Ni (nickel)) in combination or individually on polymer or other material supports. In various examples, the catalyst comprises one or more volcanic tuff modifications (e.g., one or more porous clinoptilolite minerals), is heterogeneous, and has multiple active domains.

In various examples, the catalyst (e.g., a mineral) comprises, consists, or consists essentially of one or more or all of the following metal oxide and/or metal components:

| Catalyst (e.g., mineral | |
|---|---|
| content (metal oxides)) | Range (wt %) |
| Na₂O | 0.70-1.50 |
| MgO | 0.88-2.00 |
| Al₂O₃ | 8.20-20.00 |
| SiO₂ | 50.00-70.00 |
| P₂O₅ | 0.05-0.10 |
| SO₃ | 0.04-0.09 |
| K₂O | 3.30-5.40 |
| CaO | 3.30-5.90 |
| TiO₂ | 0.30-1.20 |
| MnO | 0.02-0.08 |
| Fe₂O₃ | 3.10-6.20 |

| Catalyst (e.g., mineral | |
|---|---|
| content (metals)) | Range (ppm) |
| Sr | 1300-1800 |
| Zr | 300-500 |
| Zn | 70-110 |
| Ba | 900-1200 |
| Cu | 20-50 |
| Ni | 40-90 |
| Nb | 10-40 |
| Rb | 150-220 |
| Pb | 2-10 |
| Co | 3-12 |
| V | 50-80 |
| Y | 5-20 |
| Sn | 1-5 |
| Ga | 5-80 |

In various examples, the catalyst (e.g., a mineral) comprises one or more or all of these metal oxide and or metal components in the associated range(s) (e.g., weight percent (wt. %) and/or parts per million (ppm)).

By consisting essentially of in reference to the components of a catalyst, it is meant that an additional component or additional components do not affect the catalyst performance by more than 0.1%, more than 0.5%, more than 1%, or more than 2% relative to a catalyst operating under the same conditions and having the same composition except for the an additional component or additional components.

In various examples, the catalyst is comprises, consists, or consists essentially of one or more or all of the following mineral components:

| Mineral content (ores) | Range (wt %) |
|---|---|
| SiO₂ (α-kvartz) | 50.00-70.00 |
| Feldspar | 10-30 |
| Illite | 6.00-20.00 |
| Clinoptilolite | 10.00-30.00 |

In various examples, the catalyst comprises, consisting, or consists essentially of one or more or all of these mineral components in the associates range(s) (e.g., weight percent (wt. %)).

In various examples, the metals and/or metal oxides of the catalyst are disposed in an aluminosilicate. Without intending to be bound by any particular theory, in the case where the catalyst comprises Al₂O₃ and/or TiO₂, Al₂O₃ is desirable because of its interlayer serves as electron transfer inhibitor and/or it is considered that TiO₂ is desirable because of its chemical and/or thermal stability.

The catalyst may be an electrode (e.g., a cathode and/or an anode) in an electrochemical system. In an example, an electrode of an electrochemical system comprises a catalyst material of the present disclosure.

The catalyst is under a potential. The catalyst may be under a negative bias. Cathodic response is observed at −0.3 V vs. Ag/AgCl and product evolution occurs at appreciable rates at −2.0 V. The system may operate at voltages as high as −10 V, resulting in rapid product formation.

The methods can produce various reduction products (e.g., carbon source reduction products and, optionally, water oxidation products). Without intending to be bound by any particular theory, it is considered the potential (e.g., negative bias) the catalyst material is under can be regulated to obtain desired reduction product(s) (e.g., carbon source reduction product(s)). Examples of reduction products, which may be carbon source reduction products, include, but are not limited to, hydrogen, oxygen, organic compounds (e.g., alkanes such as, for example, pentane, hexane, alkenes such as, for example, 1-pentene, 1,3-cyclopentadiene, aromatic compounds such as, for example, benzene, alcohols, such as for example, methanol, ethanol, aldehydes such as, for example, formaldehyde, acetaldehyde, propanal, 2-propenal, butanal, 2-butenal, octanal, benzaldehyde, ketones such as, for example, 2-propanone, 3-buten-2-one, 2-cyclopentan-1-one, 2-cyclopentan-1-one, 2-pentanone, 2-butanone, oxetane, ethers such as, for example, oxirane, 2-methyl-1,3-dioxolane, 2,5-dihydrofurane, carboxylic acids and salts thereof such as formic acid, acetic acid, esters such as, for example, ethyl acetate, isomers thereof, analogs thereof, substituted analogs thereof, halogenated analogs (e.g., chlorinated analogs) thereof, and combinations thereof. Reduction products can be linear compounds, branched compounds, cyclic compounds, or combinations thereof. Reduction products can be gases and/or liquids (e.g., gases and liquids at STP or under reaction conditions).

In the case where the substrate (e.g., carbon source, such as, for example, carbon dioxide, carbonate, cyanide, and the like), hydrogen sulfide, nitrate, phosphate, sulfates, or a combination thereof), and a chloride source such as, for example, a chloride salt (e.g., sodium chloride (NaCl)), is contacted with a catalyst under an electrochemical potential (e.g., a negative bias), such that reduction products (e.g., carbon source reduction products such as, for example, graphite reduction products, carbon dioxide reduction products and/or carbonate reduction products) are formed, chloride oxidation products (e.g., hypochlorite ions) are formed. In this case, chlorinated substrate reduction products (e.g., chlorinated carbon source reduction products) are formed. Examples of chlorinated carbon source reduction products include, but are not limited to, chlorinated organic compounds comprising one or more chloride substituents. Chlorinated carbon source reduction products can be observed in the product resulting from the contacting by methods known in the art. For example, chlorinated carbon source reduction products are observed in the product by gas chromatography.

Chlorinated carbon source reduction products (e.g., a mixture of chlorinated carbon source reduction product(s) and chloride oxidation products (e.g., hypochlorite ions such as, for example, sodium hypochlorite)) may be used as bleaching or whitening agents, or stain removal agents, or in similar applications. The products can be a less expensive alternative to conventional stain removal agents. Stain removal tests showed that the mixture can remove organic stain/spots on clothing materials, such as, for example, cloths (e.g., stains/spots on cloths from fruits and other foods), without undesirable effects to the original materials color(s). The product can also be used as a biocide for removing microorganisms/bacteria in cooling towers.

The chlorinated compounds may comprise one or more chloride substituents on carbon atom(s) of the compounds (e.g., one or more Cl—C bonds). The chlorinated compounds may be long-chain (e.g., C₂ to C₁₂) chlorinated compounds. Non-limiting examples of chlorinated carbon source reduction products include, the following:

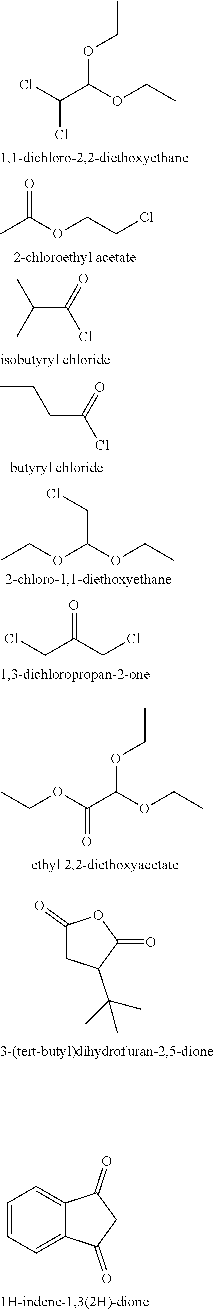

Reduction products may be separated from the reaction mixture comprising reducible material(s) and catalyst. Accordingly, in an example, a method further comprises separation of one or more reduction products. Reduction products can be separated by methods or processes known in the art. For example, reduction products are separated by dividing a system into a cathode compartment and an anode compartment. It may be desirable to have a system with a separate anode compartment and a cathode compartment because anodic oxygen is with the cathode products may cause difficulty in separation of cathodic products.

The steps of the method described in the various embodiments and examples disclosed herein are sufficient to carry out the methods of the present disclosure. Thus, in an embodiment, a method consists essentially of a combination of the steps of the methods disclosed herein. In another embodiment, a method consists of such steps.

In an aspect, the present disclosure provides systems for electrochemical reduction of carbon dioxide. The systems comprise catalyst materials (e.g., volcanic tuff modifications such as, for example, clinoptiloltite-quartz materials). A system can carry out a method of the present disclosure.

The system is an electrochemical system for reducing various substrates, for example, carbon substrates, such as, for example, graphite and carbonates, and producing organic substances. This system can also be used to reduce and/or convert substrates such as, for example, cyanide (—CN), nitrates, phosphates, sulfates to form, amines, phosphines, sulfides (mercaptans), respectively. In various examples, the system also produces oxidation products (e.g., oxygen).

For example, a system contains a batch reactor or a continuous flow reactor with a unique electrode design and catalytic component that receives electricity from DC power supply.

The reactor does not need to directly use CO₂ as a carbon source and instead can use carbonate as a carbon source. For example, CO₂ can be converted into carbonate via bubbling through alkaline solution. Alternatively, the CO₂ can be dissolved in an ionic liquid to provide high conductivity.

The system comprises an anode and a cathode. The anode and cathode can be in the same or different compartments. For example, the system can comprise a separator (e.g., an ion exchange membrane) that separates the anode and cathode. One or more electrode (e.g., cathode, anode, working electrode, or a combination thereof) can comprise the catalyst material (e.g., tuff mineral). The catalyst material may be both an electrode and a catalytic material.

The system can comprise a conventional two-compartment electrolysis system. The system can be a 2-electrode or 3-electrode system. In various examples, the system can be configured to independently produce and, optionally, collect the reduced and oxidized products. The system can comprise a bulk electrolysis system. The system can comprise an H-cell design.

For example, the system comprises a graphite electrode and the electrolyte is an aqueous sodium chloride solution. In this case, due to potential and catalyst effects, the graphite electrode acts as a sacrificial carbon source and once activated, reacts with chloride anion. Established and existing routes to haloalkanes via radical-chain reactions are difficult to control. In the instant system, the potential can be regulated to obtain desired product(s).

The system can reduce sulfate into H₂S (hydrogen sulfide). The system is also capable of incorporating the chloride ion from NaCl solutions into organic substances, with evidence of the formation of chloroform and CCl₄, as identified by GC/FID.

In an example, a system is configured for production of chlorinated compounds. In such systems, the distance (e.g., the shortest linear distance) between the anode and cathode that are connected by the catalyst or catalyst material is 0.8-1.2 cm, including all 0.01 cm values and ranges therebetween (e.g., 0.8, 0.9, 1.0, 1.1, or 1.2 cm). In an example, the distance is 1 cm.

The following Statements provide non-limiting examples of methods and systems of the present disclosure:

Statement 1. A method for electrochemical reduction of a substrate as described herein (e.g., a carbon source (e.g., carbon materials, carbon dioxide, or carbonate materials)) comprising: a) contacting a substrate (e.g., a carbon source such as, for example, carbon monoxide, carbonate, graphite, and the like, and combinations thereof) and, optionally, a chloride salt (e.g., NaCl) with a catalyst described herein (e.g., comprising Fe (iron), for example, at 2-15 weight %, or 3-7 weight %, or 4-6 weight %, and Ti (titanium), for example, at 0.3-5 weight % or 0.4-0.8 weight %, or 0.5-0.7 weight %, Ni (nickel), and Zn (zinc) disposed in an aluminosilicate (e.g., one or more volcanic tuff modifications such as, for example, clinoptiloltite-quartz materials), where the catalyst is under an electrochemical potential (e.g., a negative bias), such that one or more reduction products (e.g., carbon dioxide and/or carbonate and/or graphite reduction products, or a combination thereof) are formed, and b) optionally, separating the reduction product(s) (e.g., carbon dioxide and/or carbonate and/or graphite reduction products, or a combination thereof) from the substrate(s) (e.g., carbon monoxide and/or carbonate and/or graphite) and catalyst.

Statement 2. A method according to Statement 1, where the catalyst is based on various porous volcanic tuff material.

Statement 3. A method according to Statement 1, where the catalyst is a polymer material further comprises one or more metals and/or metal oxides, for example, other than Fe (iron), and Ti (titanium), Ni (nickel), or Zn (zinc) as described herein (e.g., Ga (gallium), Zirconium (Zr), Copper (Cu), and Vanadium (V)).

Statement 4. A method according to any one of the preceding Statements, where the catalyst is an anode and/or a cathode in an electrochemical cell or disposed between and in electrical contact with a cathode and an anode of an electrochemical cell.

Statement 5. A method according to any one of the preceding Statements, where the catalyst is a cathode and a carbon material (e.g., graphite) is an anode of an electrochemical cell.

Statement 6. A system for electrochemical reduction of a substrate (e.g., a carbon source such as, for example, a carbon material, carbon dioxide, or a carbonate material) comprising: a DC power supply; and a reactor (e.g., a batch reactor or a continuous membrane reactor) including an electrode that includes a catalytic component comprising a catalyst or catalyst material disclosed herein (e.g., a catalyst or catalyst material comprising comprising Fe (iron) at 2-15 weight %, or 3-7 weight %, or 4-6 weight %, and Ti (titanium) at 0.3-5 weight %, or 0.4-0.8 weight %, or 0.5-0.7 weight %, Ni (nickel), and Zn (zinc) disposed in an aluminosilicate (e.g., one or more volcanic tuff modifications such as, for example, clinoptiloltite-quartz materials) in electronic communication with the DC power supply.

Statement 7. A system for electrochemical reduction of the carbon source according to Statement 6, where the batch reactor includes a jacket (e.g., a jacket configured to maintain a temperature from 60-70° C.).

Statement 8. A system for electrochemical reduction of the carbon source according to Statement 6 or 7, where the reactor is connected to a water spray jet.

Statement 9. A system for electrochemical reduction of the carbon source according to any one of Statements 6-8, where an upper part of the batch reactor is configured to collect one or more product gas mixtures, and where the batch reactor defines a gas outlet.

Statement 10. A system for electrochemical reduction of the carbon source according to any one of Statements 6-9, where the electrode includes an anode and a cathode, and where the anode and the cathode are connected to each other by the catalytic component.

Statement 11. A system for electrochemical reduction of the carbon source according to any one of Statements 6-10, where the electrode includes a graphite semi-cone piece and insulated rods.

Statement 12. A system for electrochemical reduction of the carbon source according to any one of Statements 6-11, where a plurality (e.g., two or three) of the graphite semi-cone piece are connected in series.

Statement 13. A system for electrochemical reduction of the carbon source according to any one of Statements 6-12, where the catalytic component is a porous volcanic tuff based material.

Statement 14. A system for electrochemical reduction of the carbon source according to any one of Statements 6-12, where the catalytic component is a polymer material comprising Ga (gallium), Fe (iron), and Ti (titanium), Zn (zinc), and Ni (nickel).

Statement 15. A system for electrochemical reduction of the carbon source according to any one of Statements 6-14, where the catalytic component is an anode and/or a cathode in an electrochemical cell.

Statement 16. A system for electrochemical reduction of the carbon source according to any one of Statements 6-15, where the catalytic component is a cathode and a carbon material (e.g., graphite) is an anode of an electrochemical cell.

The following examples are presented to illustrate the present disclosure. They are not intended to limiting in any matter.

### Example 1

This example provides a description of electrochemical reduction of carbon dioxide and systems for electrochemical reduction of carbon dioxide.

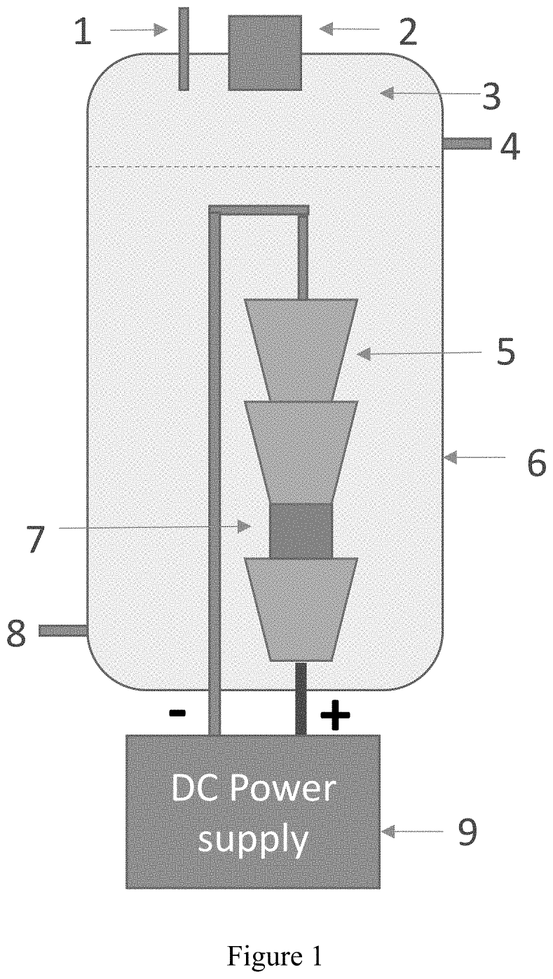

Described is an electrochemical system to reduce CO₂ into value-added organic products. Furthermore, it can be applied to the reduction of mono- or polyatomic electrolytes. One prototype of this system contains a batch reactor (6) with a special electrode that receives electricity from a DC power supply (9) (FIG. 1). A cooling jacket (inlet (8), outlet (4)) prevents overheating and helps to maintain stable temperatures of 60-70° C. The upper part (3) of the reactor is arranged for collecting the product gas mixtures, and a gas outlet (1) is designed to transfer the gas mixture for collection/analysis. An electrolyte solution is poured into the reactor lid (2). The electrode design differs from commonly used electrolysis schemes. The anode and cathode are operating as unique system as seen in FIG. 1. They are attached to each other via the mineral (catalytically active porous tuff mineral) (7). The electrode consists of a semi-cone shaped graphite (5), and rods (as a part of circuit) for holding them together. The rods are insulated to prevent solution contact. The electrode is designed based on bottom semi-cone graphite, the mineral, and two or three semi-cone graphite pieces connected in a series. Ions in the solution interact with the graphite and the mineral surface of the electrode. The mineral (1a, 1b) is very durable and highly resistive in the DC circuit. Several materials were tested for this purpose, such as stainless steel, ceramic materials, titanium, copper, quartz, etc. to validate 1a and 1b as a desirable material. These alternative materials were not catalytically active and lasted for only a few hours under the conditions of operation. On the other hand, 1a and 1b are very durable and maintains its mass and shape for several months. An analysis of the evolved gas revealed that water splitting and anion conversion occur over time as long as DC current is supplied to the system. Several good conductive solutions can be taken as an electrolyte. If a NaCl solution is used as an electrolyte, graphite serves as a sacrificial electrode: carbon atoms, water, and chloride combine with the supplied electrons to form chlorinated methane products. This process becomes possible as a result of the synchronization effect of DC current and catalytically effective material. Based on these findings, the following general reaction for the process can be written as follows:

C_{graphite}+H₂O+Cl⁻→chlorinated organic compound (e.g., CHCl₃, CCl₄, etc.)+H₂ (trace)+O₂.

The reaction is more intensive at the boundaries of the semi-cone shaped graphite and the mineral. Considerable graphite erosion can be seen between the catalyst and graphite boundaries.

When a Na₂CO₃ solution was used as an electrolyte solution in the reactor, several organic compounds were observed based on the gas mixture GC analysis. An intense oxygen signal was observed during GC analysis, implying high oxygen concentration in the gas mixture. The other product of water splitting, hydrogen, was observed at trace concentration levels. The mismatch between hydrogen and oxygen can be rationalized by the incorporation of H₂ into hydrocarbon products and alternative cathodic processes other than proton reduction (i.e. electrolyte reduction). The following organic compounds are observed based on GC/HP-Plot/Q+PT analysis: methanol, ethanol, formaldehyde, acetaldehyde, 2-methyl-1,3-dioxolane, ethyl acetate, 2-cyclopentan-1-one, benzaldehyde, octanal, 2-propanone, oxetane, propanal, oxirane, 2-propenal, 1-pentene, 1,3-cyclopentadiene, pentane, 2-butenal, 2,5-dihydrofurane, 3-buten-2-one, butanal, 2-butanone, hexane, benzene, and 2-pentanone.

In the system, a two-compartment conventional electrolysis system was intentionally not used. Evolved oxygen from anode site is mixed with cathode products. The GC spectrum intense oxygen peak confirms the high concentration of oxygen. It was expected that an oxygen-rich medium would increase fuel capability of the gas mixture, and tested the gas mixture combustion ability. It is a burning gas mixture that combusts without uncontrollable explosions, which makes it suitable as a fuel material.

In addition, a traditional bulk electrolysis or H-cell design may be employed. In this scheme, the 1a electrocatalyst serves as the cathode, enabling reduction products to be isolated and analyzed without interference or inclusions of oxygen and other oxidation products. The counter electrode in such designs may span a suite of materials, including Pt mesh and wire for small scale electrolysis for analysis, and carbon-based electrodes for cost-effective scale-up. In such schemes, an ion exchange membrane is needed for charge balance, such as porous glass or Nafion. Whereas the mineral (1a or 1b) may serve directly as both an electrode and catalytic material, alternative designs include incorporation into modified electrodes, including composites and hybrids where the mineral is mechanically incorporated into resins, polymers, gels, layered sheets, pressed powders, and thin films where conductive materials are deposited onto CQ substrates. H-cell reactors where CQ serves as both an anodic and cathodic catalyst enable a better understanding of the role of the mineral in oxidation, where carbon can be included or excluded as a supporting electrode material.

Natural sources of CO₂ and carbonate are plentiful. In our system, atmospheric CO₂ can be supplied in the basic medium of the reactor or natural carbonate can be used for carbon sequestration.

Traditional electrolysis of Na₂CO₃ electrolyte solution yields hydrogen and oxygen volume ratio as roughly equal to 2:1, whereas in our system the amount of the hydrogen is trace (GS-MS signal shows that hydrogen concentration is lower than oxygen). The high level of oxygen indicates that the anodic current is used for water oxidation. This process evolves four electrons and four protons per molecule of O₂. As described above, the lack of significant amounts of H₂ indicate that the generation of protons and electrons does not terminate in H₂ evolution. Instead, the cathodic current is used to reduce carbonate, with protons available for the formation of hydrocarbon products. This is further supported by the non-explosive nature of the evolved gas mixture, in which a combination of H₂ and O₂ would exhibit a different combustion behavior. This lack of explosive product gases is also observed when NaCl is used as electrolyte. The existence of organic chlorine derivatives in the gas mixture limits the use of this product mixture as fuel, but the gas mixture obtained from Na₂CO₃ solution can be used as a fuel source. The simplicity and effectiveness of the system allows for the construction of a small reactor potentially as a vehicle fuel source.

A variety of strong electrolytes has been tested, and the obtained results revealed that the system tends to reduce the polyatomic anion component of many strong or moderate (ionizable) electrolytes. For example, a traditional aqueous electrolysis of Na₂SO₄ results in evolution of hydrogen and oxygen gases. However, in the system, reactive transformations of Na₂SO₄ containing electrolyte solution are different than simple electrolysis. Sulfate reduction (SO₄²⁻→H₂S) is observed based on the rotten egg odor of the evolved gas mixture. It can be inferred that the odor belongs to some sulfur containing organic species.

### Experimental Section

700 ml 4% NaCl solution was prepared, and poured into the reactor (capacity of 800 ml). 8 A current 20V voltage were supplied to the electrochemical reactor (FIG. 1). Upon switching on the DC power supply gas evolution was immediately observed. The reactor was allowed to heat up to 60-70° C., then the cooling system was turned on in order to maintain the reactor temperature stability. In a steady state condition (after 2.5 hours), 1 L gas sample was collected via a gas sampling bag. Half an hour later, the outlet gas hose was connected to the trapping tube that was immersed into a liquid nitrogen Dewar flask, and the gas mixture was collected in a liquid nitrogen trap for the next three hours. The sample bag and the trapped gas mixture were analyzed by HP 5972 GCMS (5890GC). Exact formula determination was done by high resolution GC-MS (MS-6890 GC). Content of the gas mixture: H₂, O₂, CHCl₃, and CCl₄

The same procedure was applied to the 1 L 10% Na₂CO₃ solution. The reactor capacity is 1.2 L. The outlet gas hose was connected to the trapping tube immersed into the isopropyl alcohol/dry ice bath. Condensed methanol and ethanol vapors was collected and analyzed via GC-MS and NMR (Varian-500 MHz) equipment. The gas mixture was also collected via above mentioned procedure. The obtained gas analysis revealed that it was trace hydrogen, oxygen, and the other above-mentioned organic species.

The mineral is a naturally occuring substance, and its content has been analyzed by XRD. It has mainly clinoptilolite, quartz and some mixed silicates of Na, Al, Ga, K, Ca, Mg, Fe, and Ti metals.

Electrochemical analysis is performed using a suite of 2- and 3-electrode techniques. In an H-cell designed to separate the anodic and cathodic processes, a working electrode (WE) comprising the mineral with a copper wire lead was immersed in the electrolyte. A Ag/AgCl or Ag wire quasi-reference electrode was used to measure the voltage drop to the WE. A Pt-mesh counter electrode (CE) is used in the second compartment of the H-cell. In this scheme, the voltage drops between the WE and CE was not measured and the surface area of the CE is large enough to match the current flow at the WE. The 3-electrode H-cell enabled independent collection and analysis of reduced and oxidized products. In the 2-electrode scheme, the reference lead is placed in series to the CE. In this design, the overall cell voltage was measured between the WE and CE. The electrode potentials are not directly measured, instead the overall cell voltage is controlled where the anodic and cathodic currents are matched. This design limits the overall voltage drop of the system and is useful for optimizing parameters of a bulk electrolysis cell.

### Example 2

This example provides a description of characterization of clinoptilolite-quartz materials of the present disclosure.

FIGS. 2-7 show X-ray Diffraction (XRD) spectra of examples of clinoptilolite-quartz.

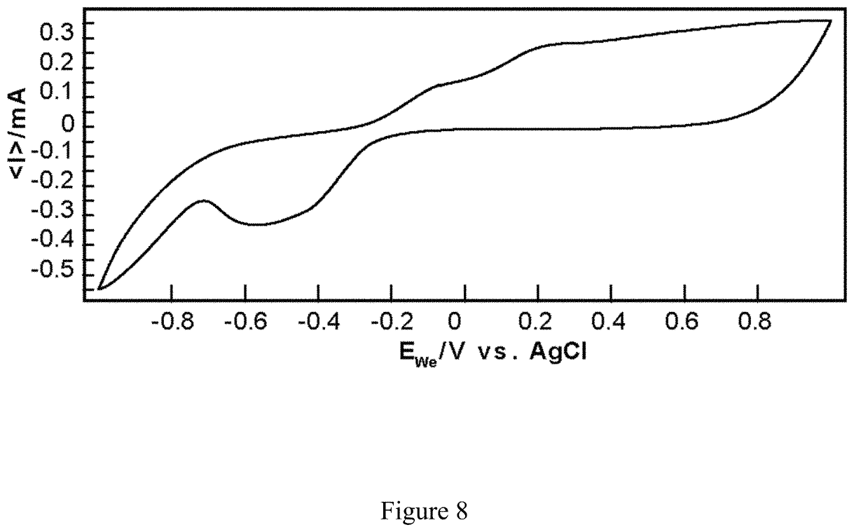

FIG. 8 shows a cyclic voltammogram in a 10% aqueous carbonate solution using green tuff as a working electrode, a Ag/AgCl reference, and a Pt-wire counter electrode in a standard one-compartment cell. An onset of cathodic current is observed at −0.3 V vs Ag/AgCl, followed by further reduction at −0.75 V vs Ag/AgCl at a scan rate of 500 mV/s.

### Example 3

This example provides a description of electrochemical reduction of graphite in the presence of a chloride source.

The system is capable of converting NaCl to sodium hypochlorite (NaOCl). In this example, only brine solution is taken. The graphite electrode became sacrificial and eroded. That provides carbon for chlorinated organic compounds GC analysis of obtained sodium hypochlorite solution after 1 to 8-hour operation time shows that some long chain-chlorinated organic compounds are also formed. Mixture of NaOCl and long chain chlorinated organic compounds can be applied as a bleach/whitening agent. It can be used as a cheaper alternative of stain removal agents. Stain removal tests showed that the mixture is good to remove organic stain/spots (e.g., stains/spots from fruits and other foods) without destroying original cloth colors. The product can be used as a biocide for removing microorganisms/bacteria in cooling towers. The following organic compounds are observed by GC analysis:

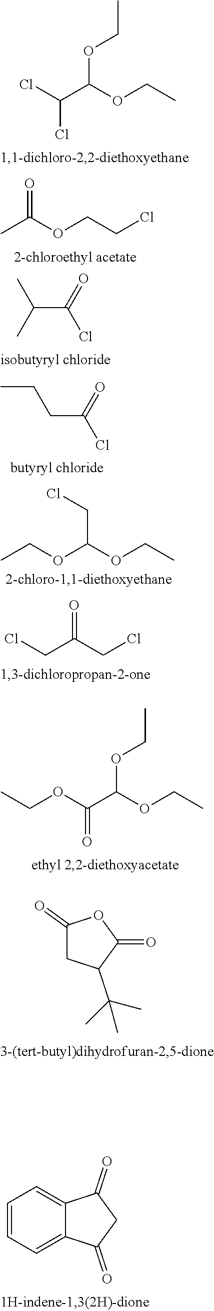

### Example 4

This example provides a description of the characterization of catalyst materials of the present disclosure.

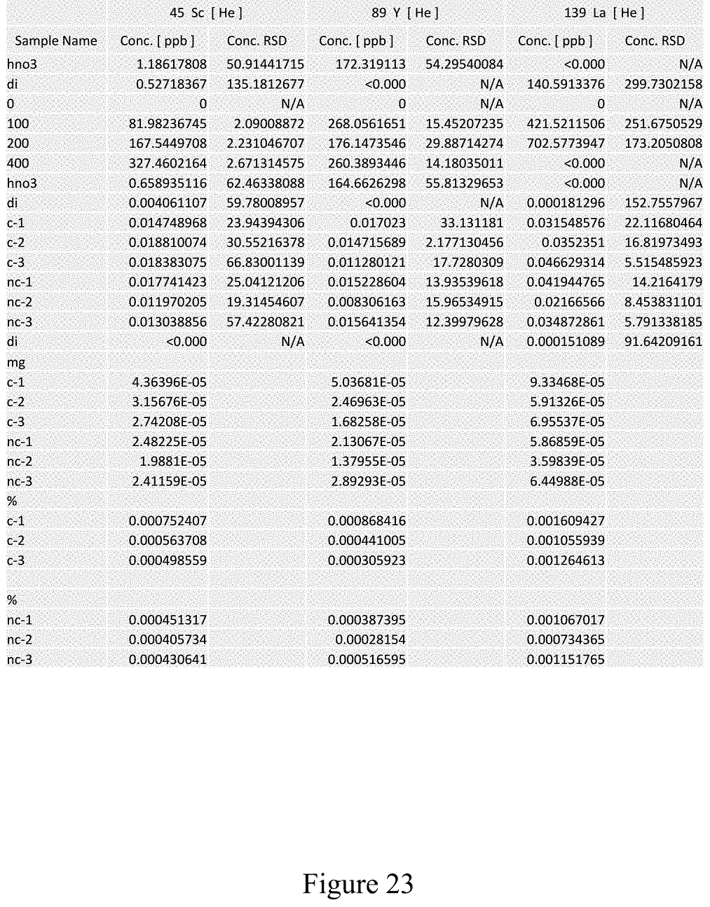

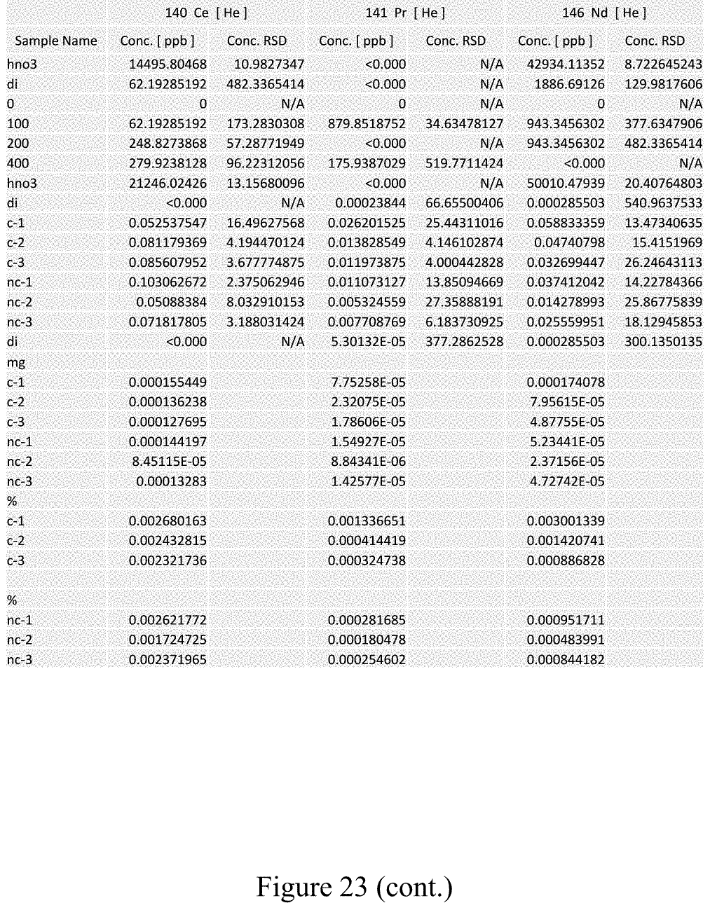

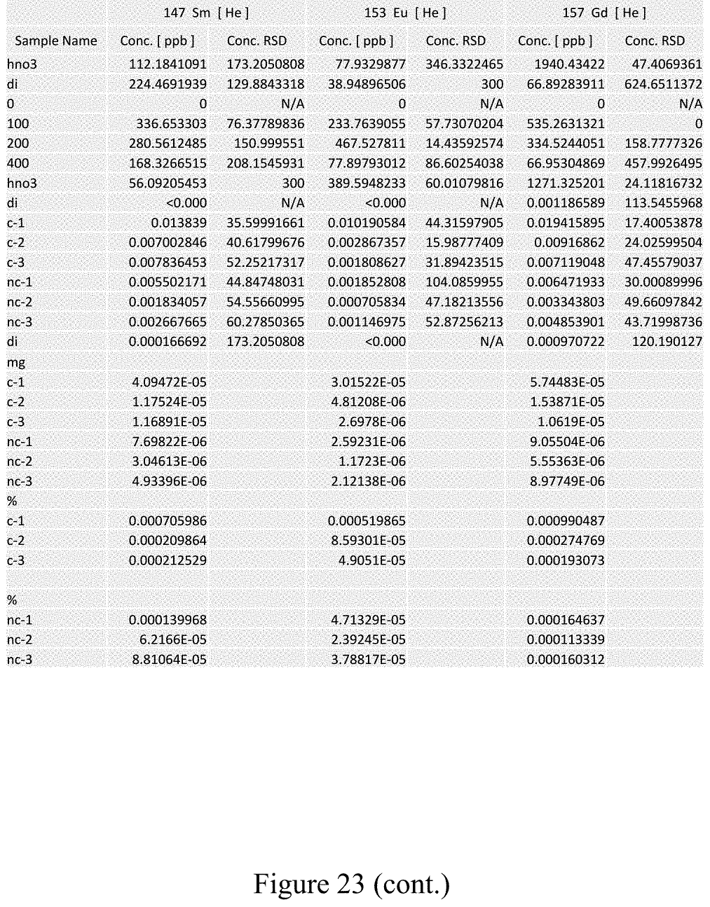

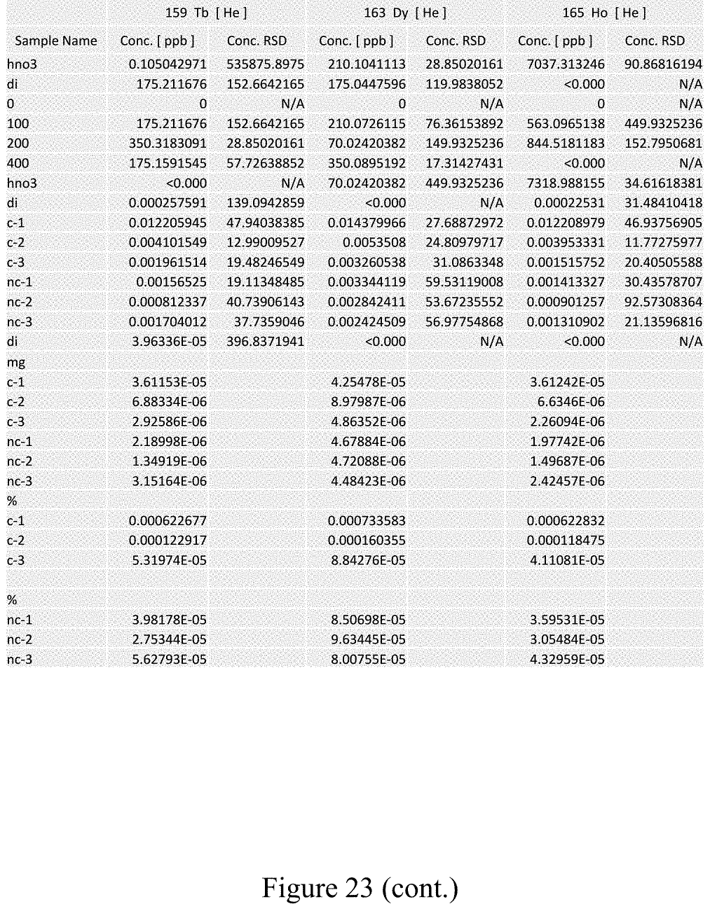

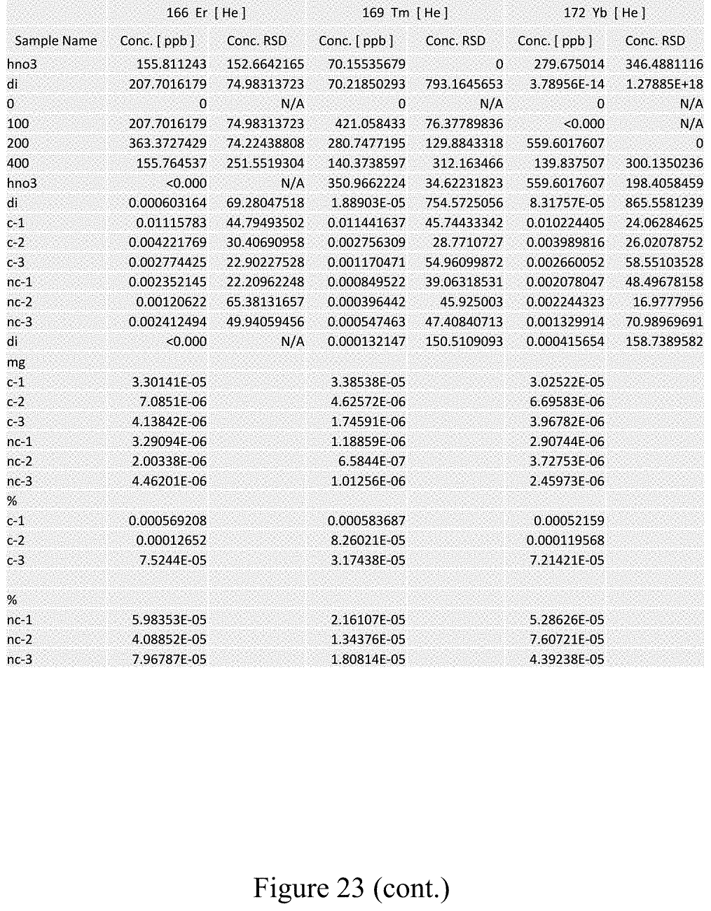

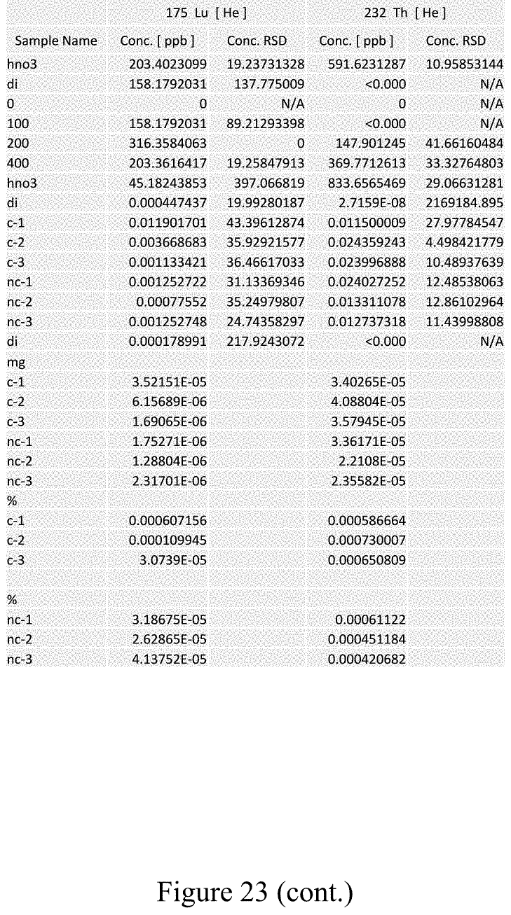

ICP-MS analysis: Samples of catalyst materials were prepared in a mixture of hydrochloric and nitric acids. FIG. 23 shows ICP/MS characterization data for various samples from Table 1.

TABLE 1
Sample information.

| | | Conical | tube + | | | |
|---|---|---|---|---|---|---|
| | | tube | solution | solution | | |
| Name | sample (mg) | (g) | (g) | (g) | Dilution | |
| c-1 | 5.8 | 12.8661 | 64.963 | 52.0969 | 0.2552 | 14.494 |
| c-2 | 5.6 | 12.8781 | 65.0332 | 52.1551 | 0.4891 | 15.7381 |
| c-3 | 5.5 | 12.9765 | 66.3381 | 53.3616 | 0.52 | 14.5357 |
| nc-1 | 5.5 | 12.9149 | 67.4104 | 54.4955 | 0.5634 | 14.4648 |
| nc-2 | 4.9 | 13.0113 | 65.8649 | 52.8536 | 0.5066 | 15.9194 |
| nc-3 | 5.6 | 12.9322 | 68.6165 | 55.6843 | 0.5275 | 17.5208 |

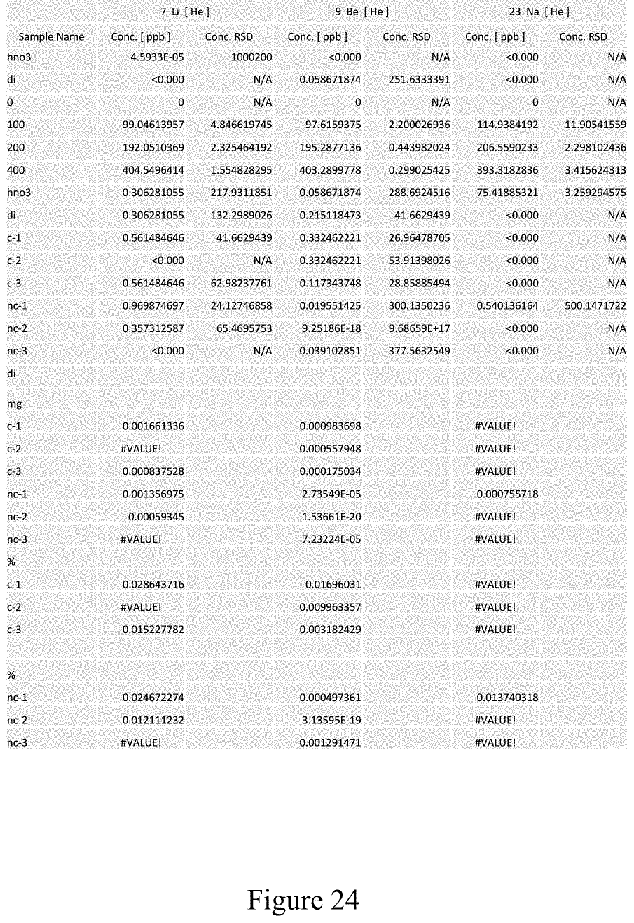

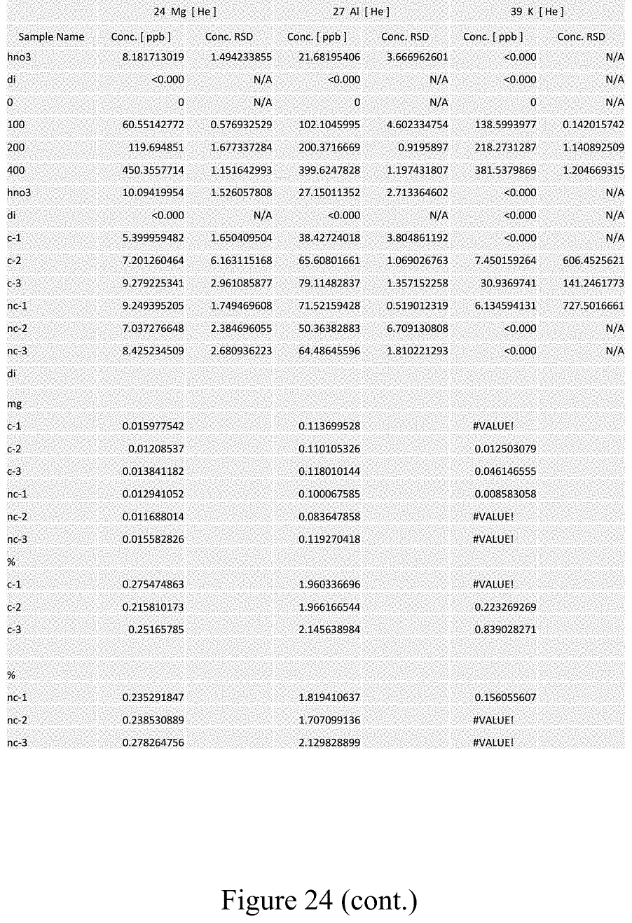

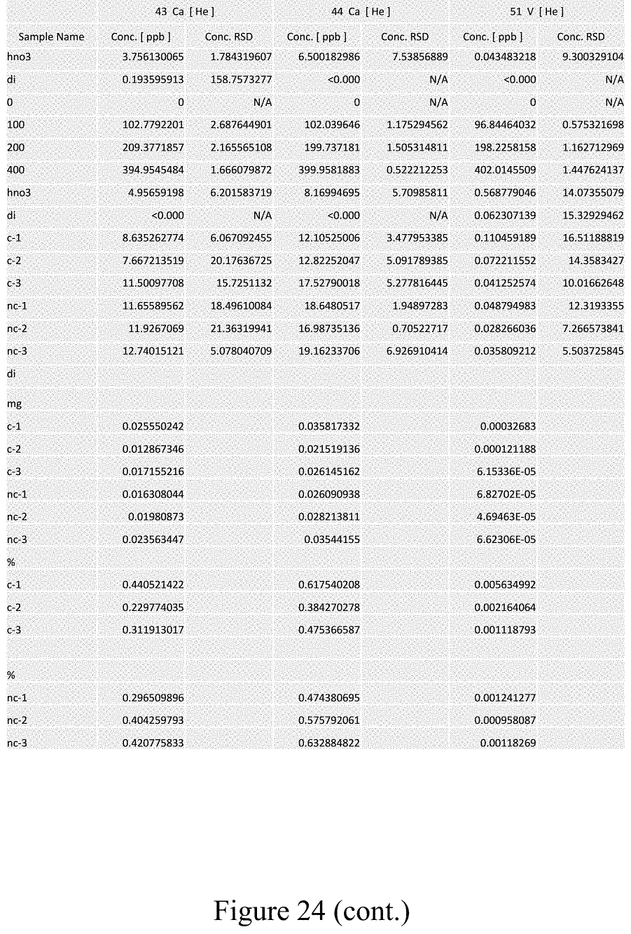

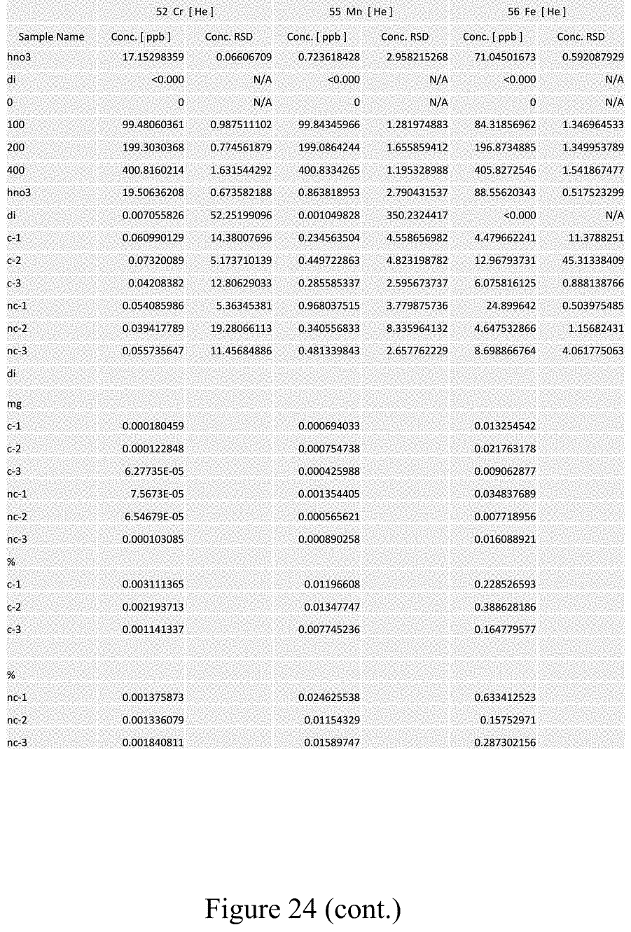

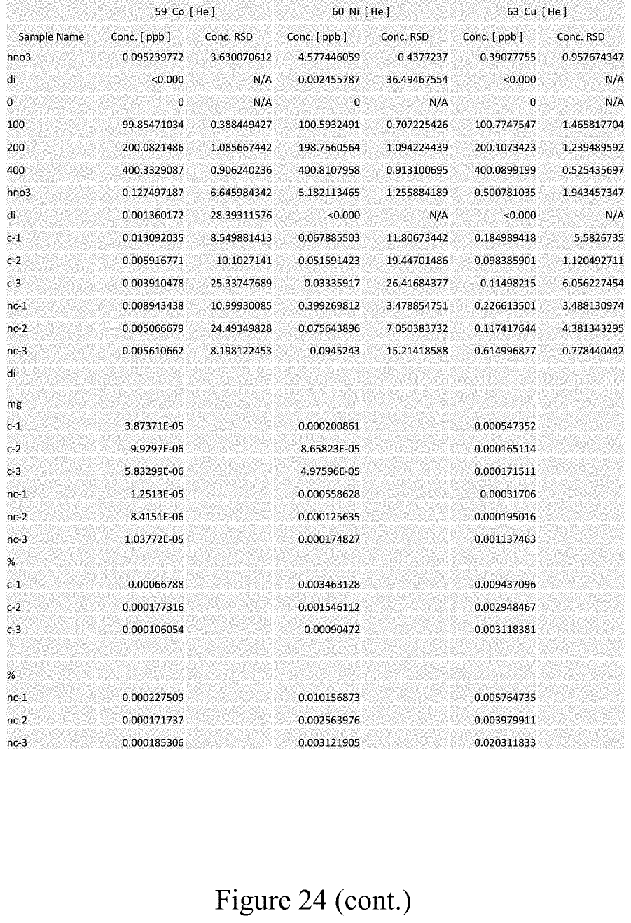

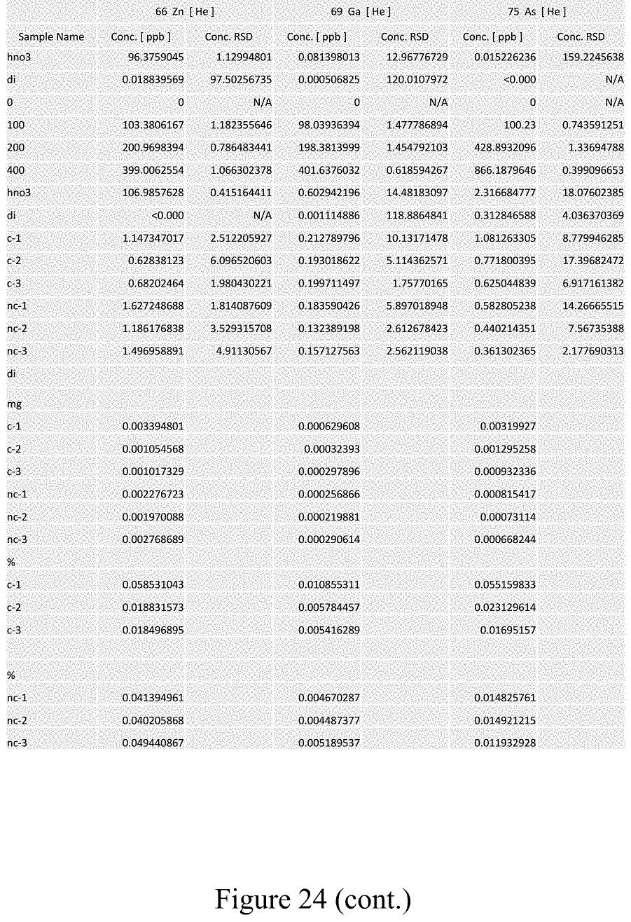

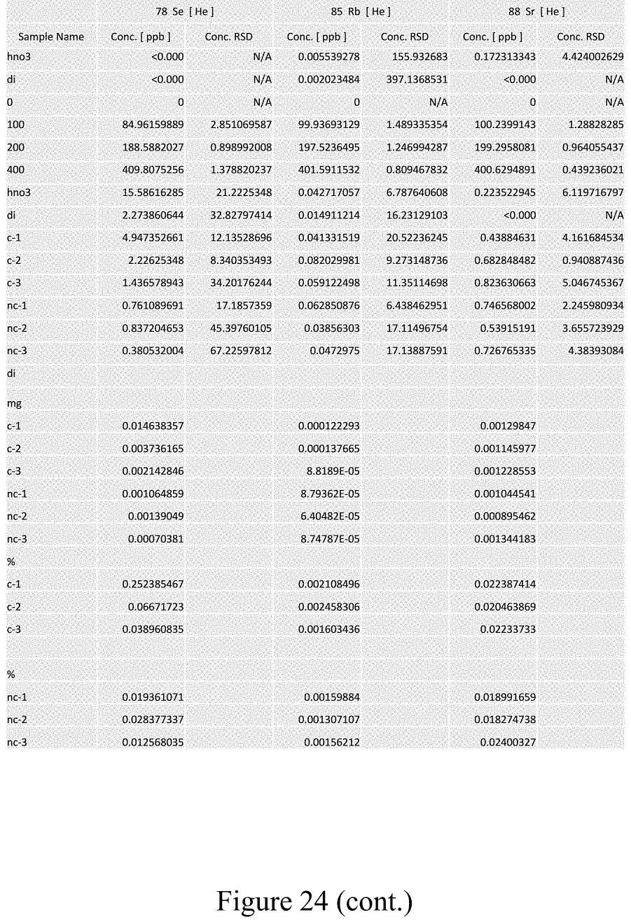

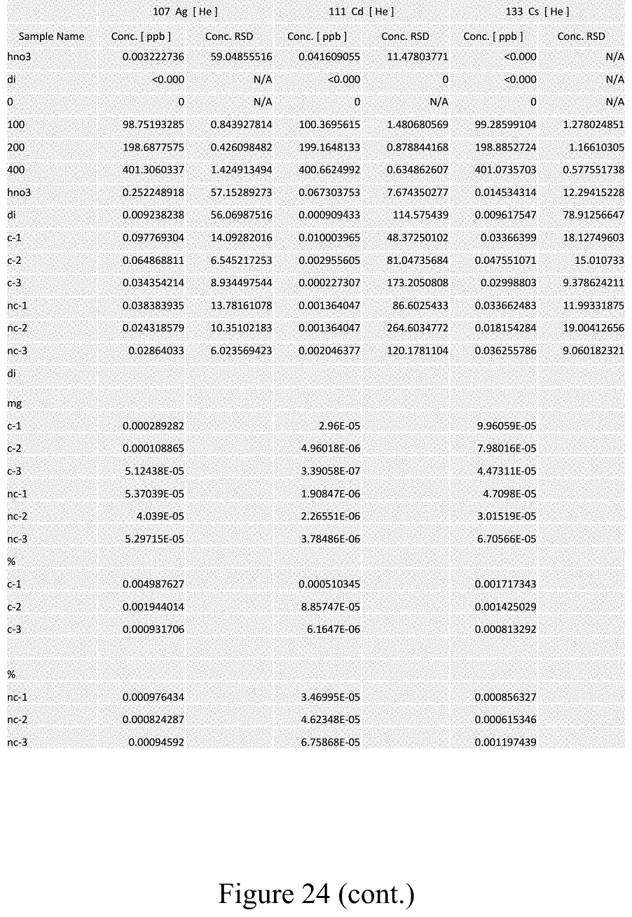

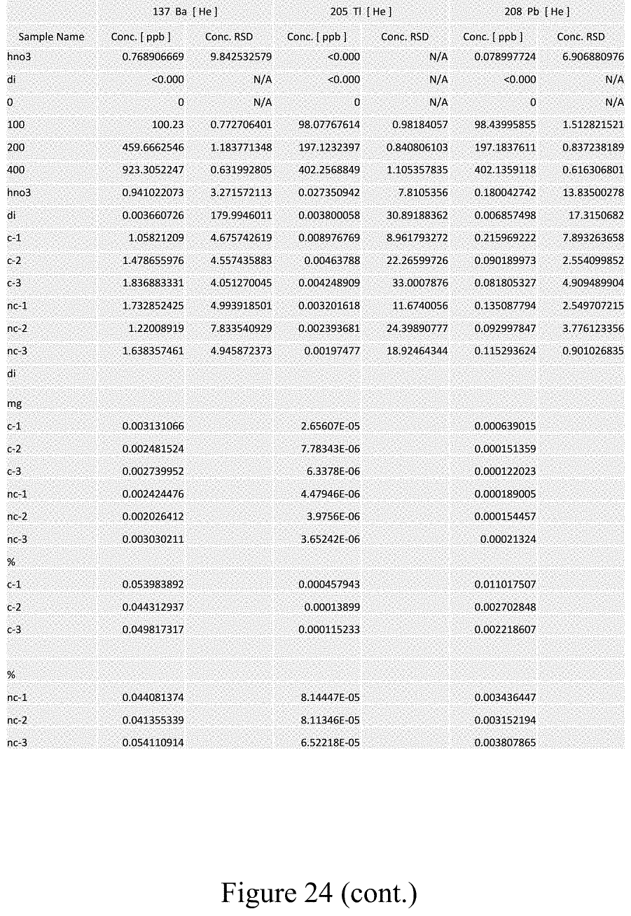

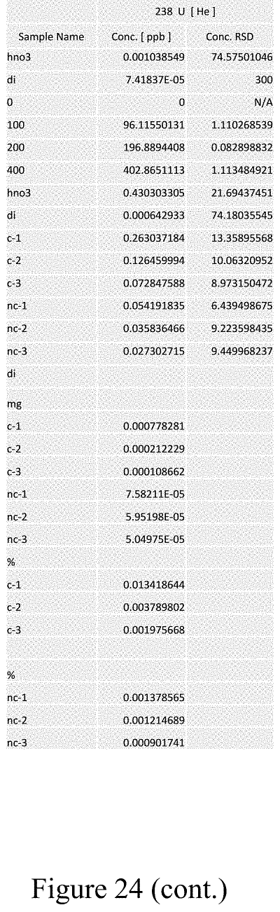

ICP-MS analysis: Samples of catalyst materials were prepared in a mixture of hydrochloric and nitric acids. FIG. 24 shows ICP/MS characterization data for various samples from Table 2.

TABLE 2
Sample information.

| | | | tube + | | | |
|---|---|---|---|---|---|---|
| | sample | Conical | solution | solution | | |
| Name | (mg) | tube (g) | (g) | (g) | Dilution | |
| c-1 | 5.8 | 12.8661 | 64.963 | 52.0969 | 0.2552 | 14.494 |
| c-2 | 5.6 | 12.8781 | 65.0332 | 52.1551 | 0.4891 | 15.7381 |
| c-3 | 5.5 | 12.9765 | 66.3381 | 53.3616 | 0.52 | 14.5357 |
| nc-1 | 5.5 | 12.9149 | 67.4104 | 54.4955 | 0.5634 | 14.4648 |
| nc-2 | 4.9 | 13.0113 | 65.8649 | 52.8536 | 0.5066 | 15.9194 |
| nc-3 | 5.6 | 12.9322 | 68.6165 | 55.6843 | 0.5275 | 17.5208 |

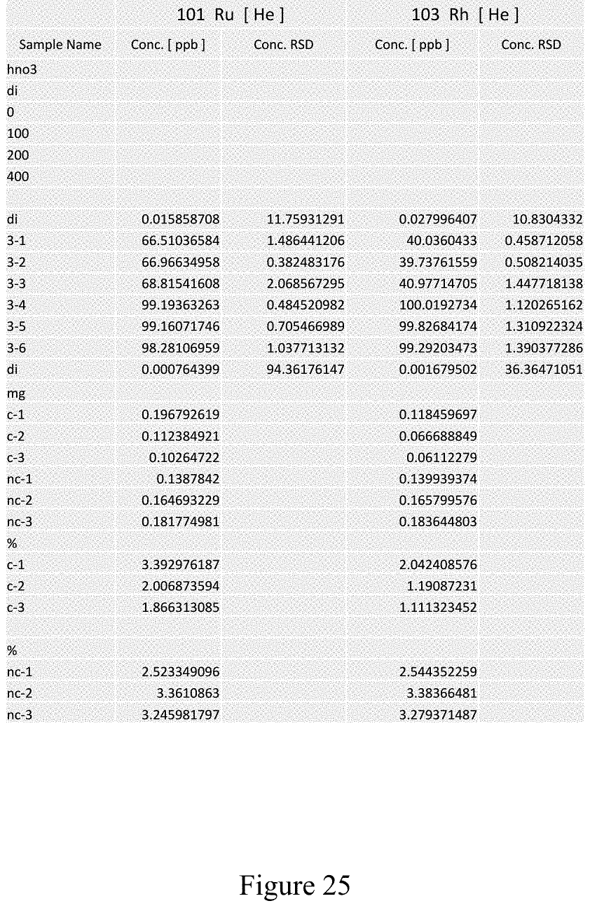

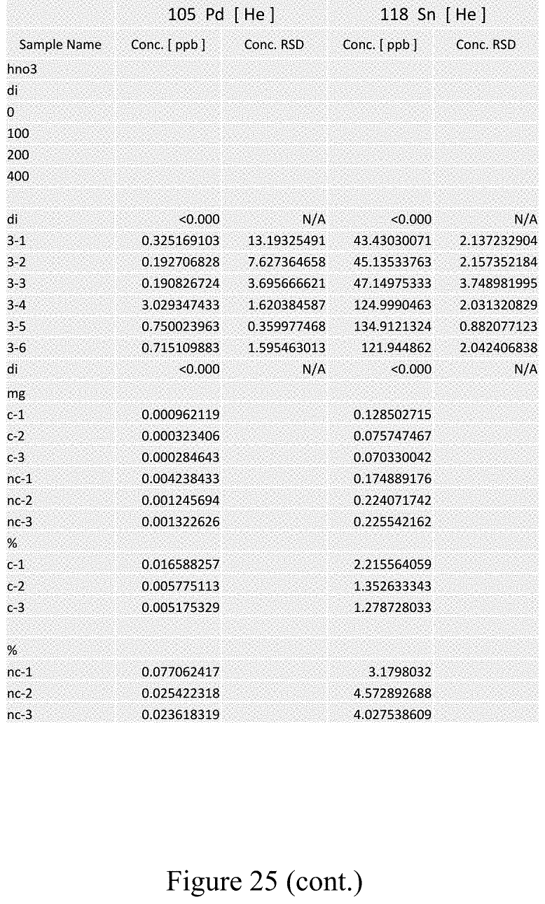

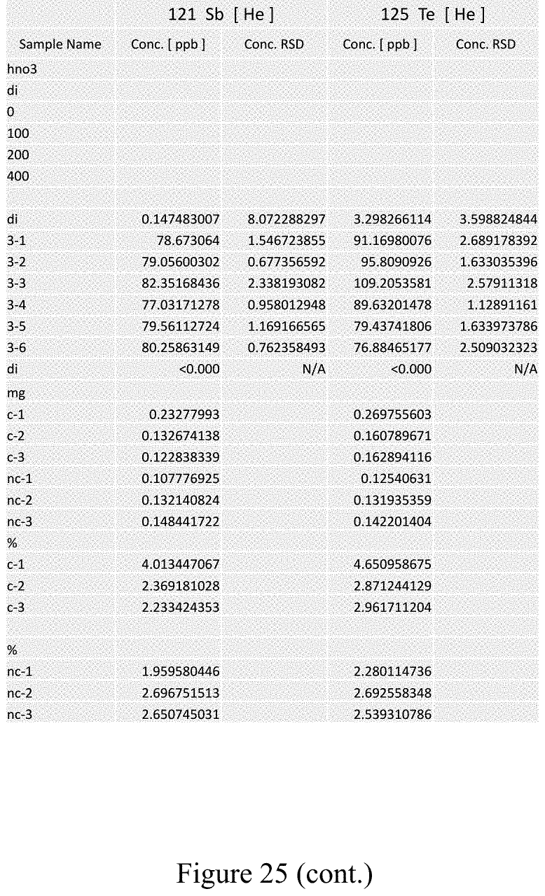

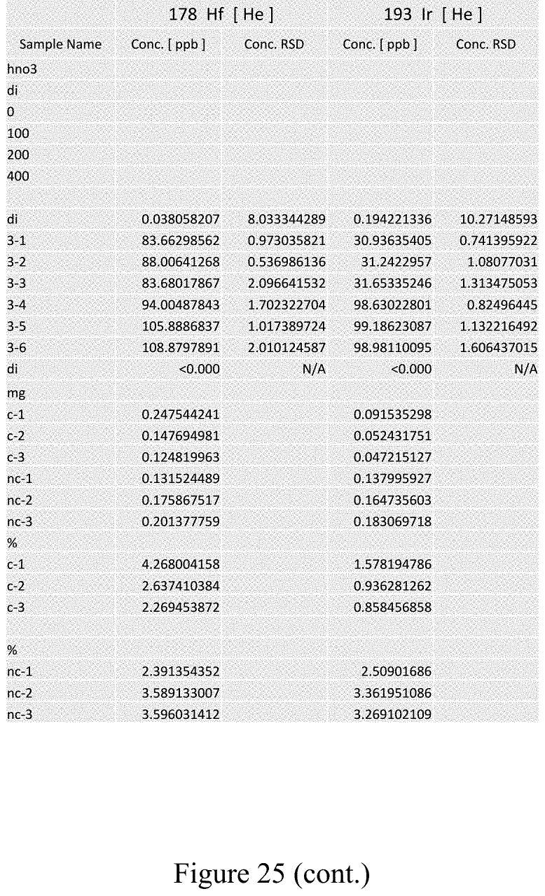

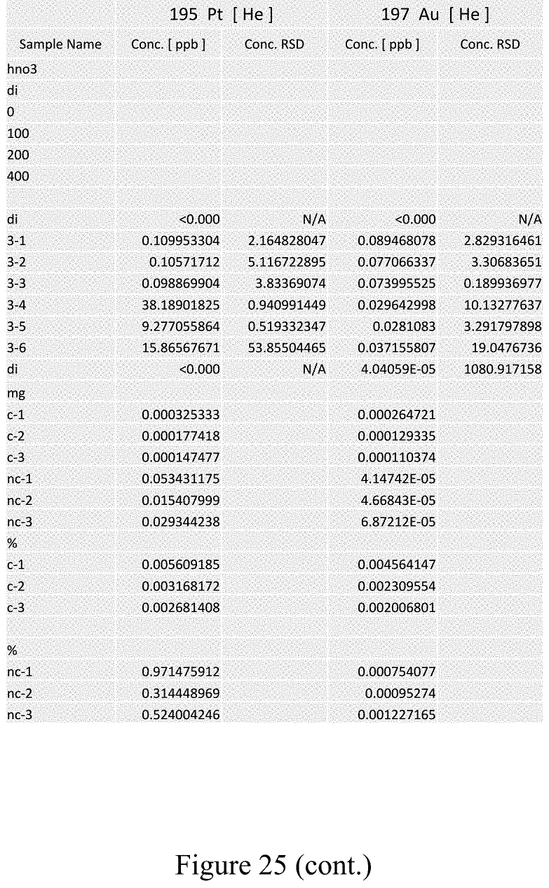

ICP-MS analysis: Samples of catalyst materials were prepared in a mixture of hydrochloric and nitric acids. FIG. 25 shows ICP/MS characterization data for various samples from Table 3.

TABLE 3
Sample information.

| | | Conical | tube + | | | |
|---|---|---|---|---|---|---|
| | sample | tube | solution | solution | | |
| Name | (mg) | (g) | (g) | (g) | Dilution | |
| c-1 | 5.8 | 12.8661 | 64.963 | 52.0969 | 0.2552 | 14.494 |
| c-2 | 5.6 | 12.8781 | 65.0332 | 52.1551 | 0.4891 | 15.7381 |
| c-3 | 5.5 | 12.9765 | 66.3381 | 53.3616 | 0.52 | 14.5357 |
| nc-1 | 5.5 | 12.9149 | 67.4104 | 54.4955 | 0.5634 | 14.4648 |
| nc-2 | 4.9 | 13.0113 | 65.8649 | 52.8536 | 0.5066 | 15.9194 |
| nc-3 | 5.6 | 12.9322 | 68.6165 | 55.6843 | 0.5275 | 17.5208 |

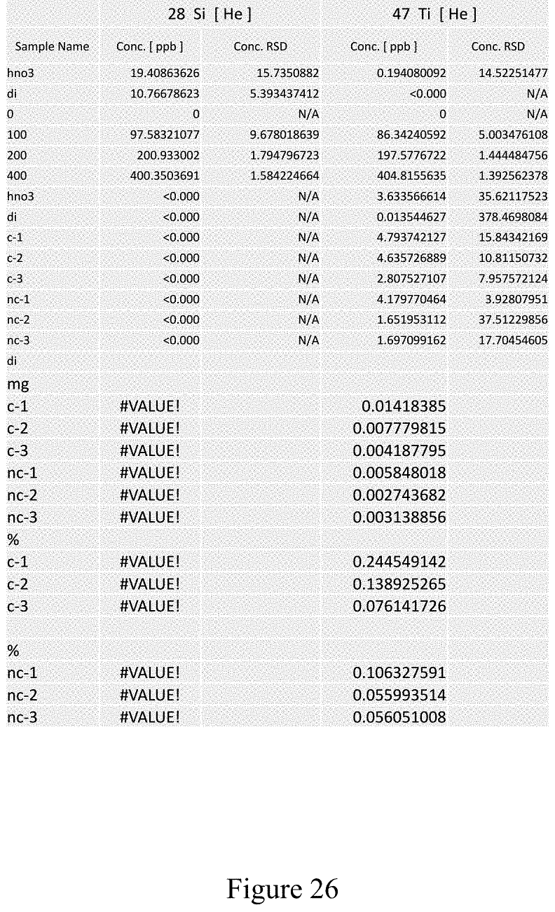

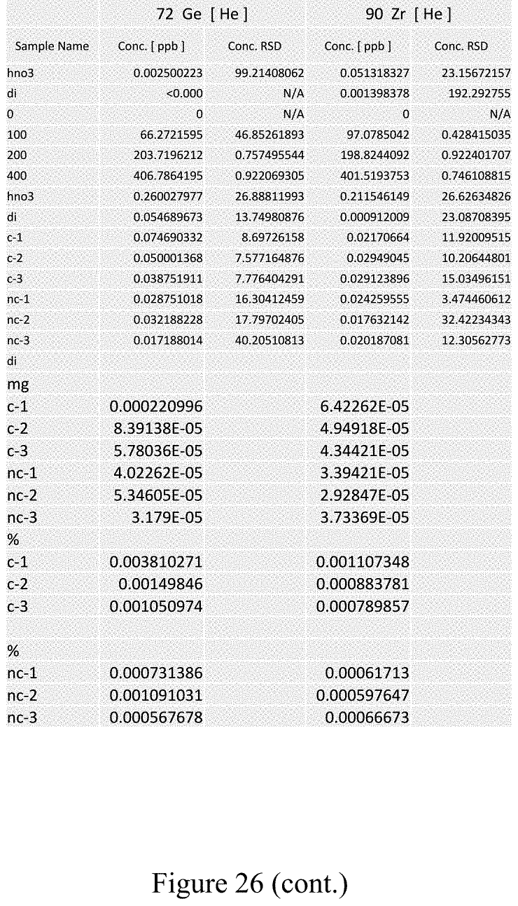

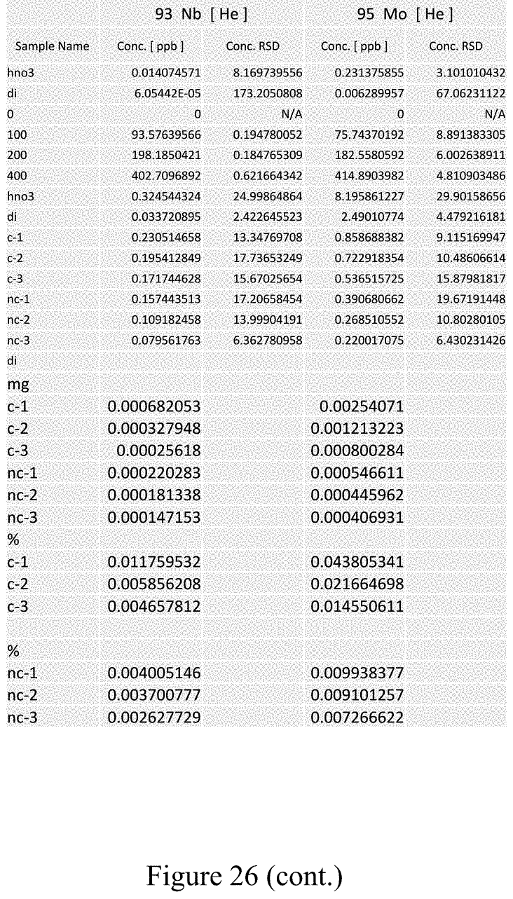

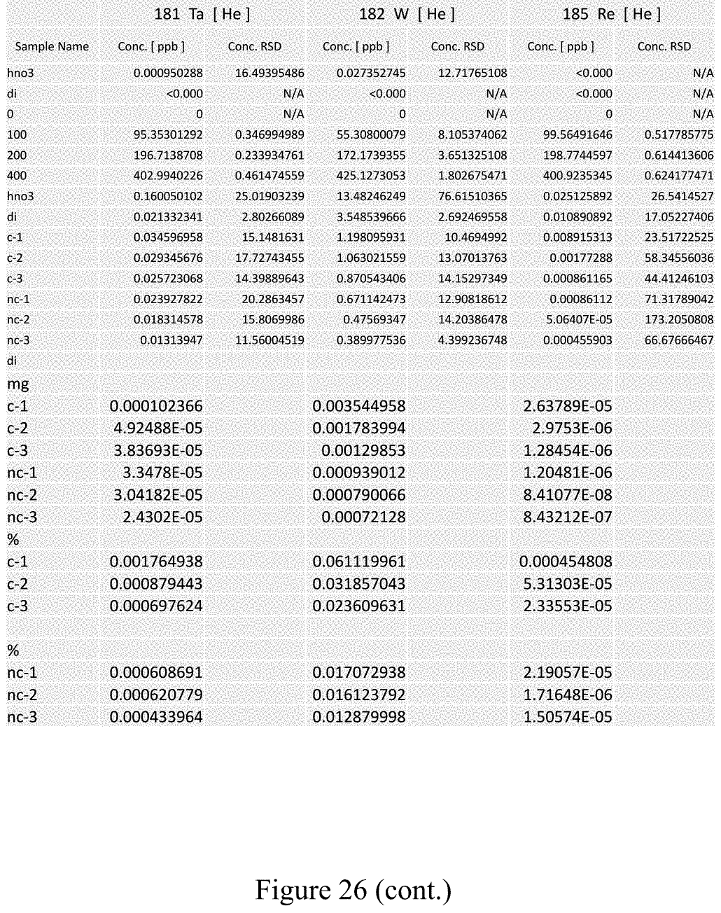

ICP-MS analysis: Samples of catalyst materials were prepared in a mixture of hydrochloric and nitric acids. FIG. 26 shows ICP/MS characterization data for various samples from Table 4.

TABLE 4
Sample Information.

| | sample | Conical | tube + solution | solution | | |
|---|---|---|---|---|---|---|
| Name | (mg) | tube (g) | (g) | (g) | Dilution | |
| c-1 | 5.8 | 12.8661 | 64.963 | 52.0969 | 0.2552 | 14.494 |
| c-2 | 5.6 | 12.8781 | 65.0332 | 52.1551 | 0.4891 | 15.7381 |
| c-3 | 5.5 | 12.9765 | 66.3381 | 53.3616 | 0.52 | 14.5357 |
| nc-1 | 5.5 | 12.9149 | 67.4104 | 54.4955 | 0.5634 | 14.4648 |
| nc-2 | 4.9 | 13.0113 | 65.8649 | 52.8536 | 0.5066 | 15.9194 |
| nc-3 | 5.6 | 12.9322 | 68.6165 | 55.6843 | 0.5275 | 17.5208 |

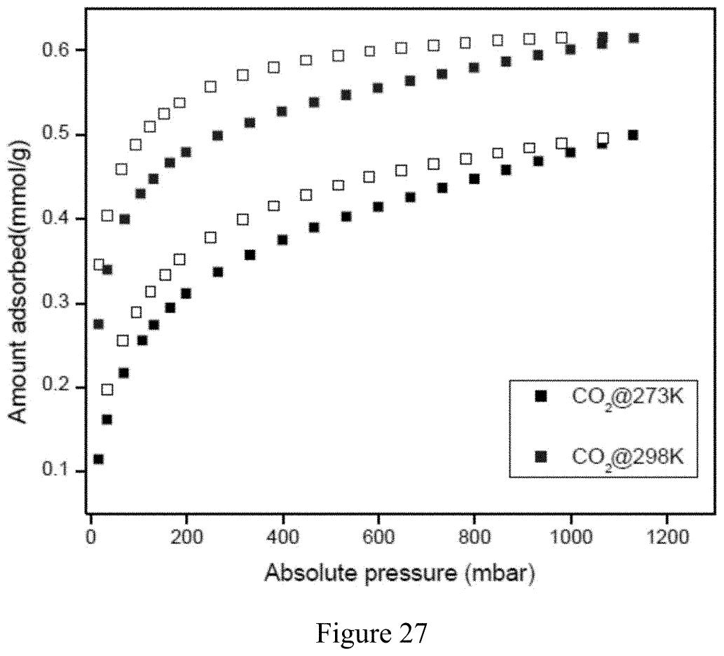

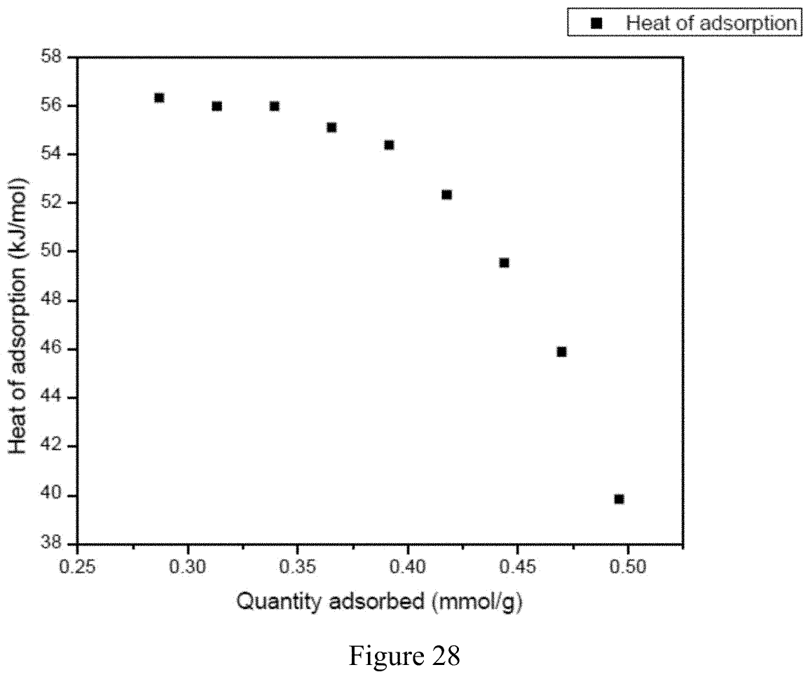

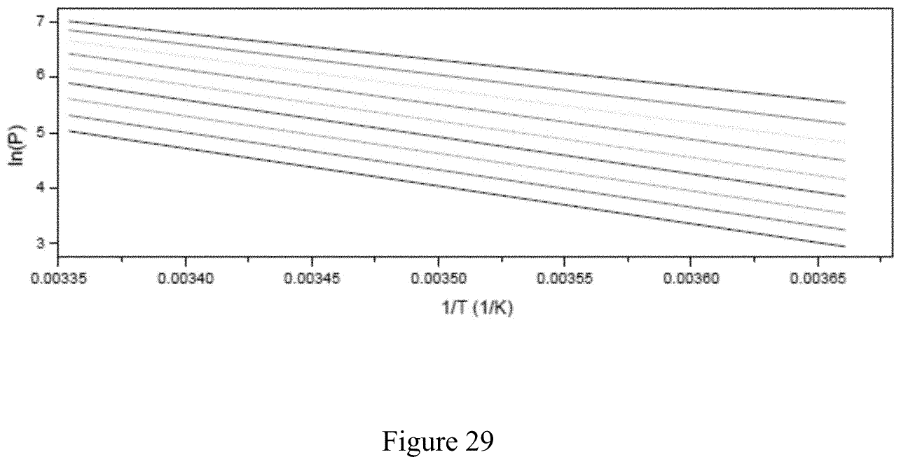

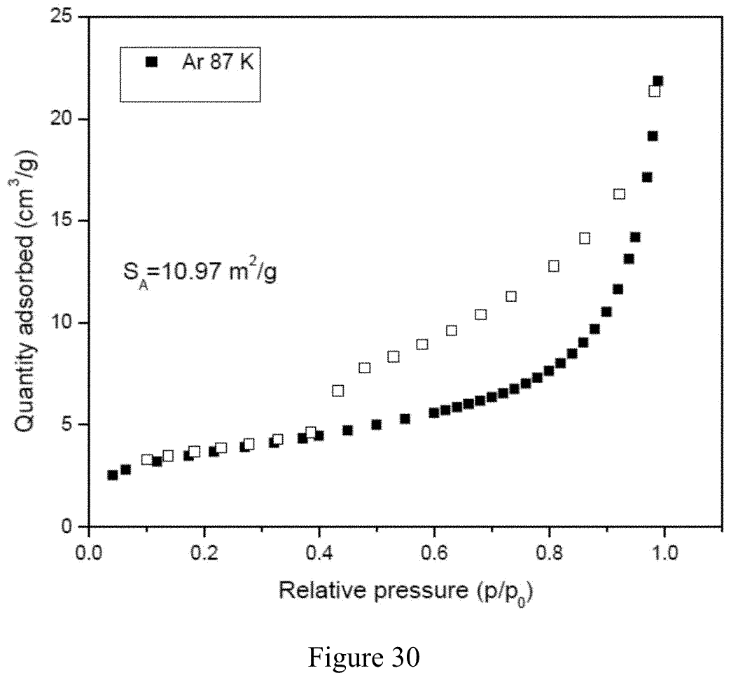

Surface study: Tuff is nonporous (SA=10.97 m²/g) mineral confirmed by BET analysis (shown in FIG. 30). Although CO₂ capacity is low (0.6 mmol/g at 1 bar) (see FIGS. 27 and 28), the interaction between tuff and CO₂ is chemisorptive (see FIG. 29). Chemisorptive interaction with CO₂ supports catalytic activity tuff in CO₂ reduction.

### Example 5

This example provides a description of the characterization of catalyst materials of the present disclosure.

The following is the elemental analysis of the two modifications of the green samples (1A and 1B). The catalyst components were identified using Wavelength Dispersive X-Ray Fluorescence (WDXRF) Spectrometer (Model: S8 TIGER, BRUKER).

Sample main content, wt %

| Sample | | | | | | |
|---|---|---|---|---|---|---|
| name | Na₂O | MgO | Al₂O₃ | SiO₂ | P₂O₅ | SO₃ |
| 1A | 1.05 | 1.47 | 12.58 | 62.02 | 0.07 | 0.07 |
| 1B | 1.09 | 1.36 | 13.42 | 60.14 | 0.05 | 0.08 |

| Sample | | | | | | | |
|---|---|---|---|---|---|---|---|
| name | K₂O | CaO | TiO₂ | MnO | Fe₂O₃ | Cl⁻ | HTML* |
| 1A | 4.66 | 4.93 | 0.63 | 0.04 | 4.48 | 0.04 | 7.8 |
| 1B | 4.95 | 4.59 | 0.60 | 0.04 | 4.49 | 0.16 | 8.4 |

*HTML - high temperature (950° C.) mass loss.

Sample minor content (in ppm)

| Sample | | | | | | | |
|---|---|---|---|---|---|---|---|
| name | Sr | Zr | Zn | Ba | Cu | Ni | Nb |
| 1A | 1465 | 384 | 85 | 1084 | 30 | 67 | 27 |
| 1B | 1120 | 396 | 96 | 1062 | 34 | 78 | 18 |

| Sample | | | | | | |
|---|---|---|---|---|---|---|
| name | Pb | Co | V | Y | Sn | Ga |
| 1A | 5 | 9 | 64 | 12 | 2 | 51 |
| 1B | 4 | 8 | 78 | 9 | 3 | 65 |

Sample mineralogical content, wt %

| Sample | SiO₂ | | | |
|---|---|---|---|---|
| Name | (α-kvartz) | Feldspar | Illite | Clinoptilolite |
| 1A | 58 | 16 | 8 | 18 |
| 1B | 50 | 25 | 10 | 15 |

1. A method for electrochemical reduction of a carbon source comprising:
contacting a carbon source and, optionally, a chloride salt, with a catalyst comprising Fe (iron) at 2-15 weight %, and Ti (titanium) at 0.3-5 weight %, Ni (nickel), and Zn (zinc) disposed in an aluminosilicate, wherein the catalyst is under an electrochemical potential, such that a carbon-source, reduction product is formed, and
optionally, separating the carbon-source, reduction product from the catalyst.

2. The method of claim 1, wherein the carbon source is carbon dioxide or a carbonate material.

3. The method of claim 1, wherein the catalyst is based on various porous volcanic tuff material.

4. The method of claim 1, wherein the catalyst further comprises one or more of Ga (gallium), Zirconium (Zr), Copper (Cu), and Vanadium (V).

5. The method of claim 1, wherein the catalyst is an anode and/or a cathode in an electrochemical cell or disposed between and in electrical contact with a cathode and an anode of an electrochemical cell.

6. The method claim 1, wherein the catalyst is a cathode and a carbon material is an anode of an electrochemical cell.

7. The method of claim 1, wherein the carbon-source, reduction product is chosen from carbon monoxide, hydrogen, organic compounds, chlorinated organic compounds, chloride oxidation products, and combinations thereof.

8. The method of claim 7, wherein the chloride oxidation products are hypochlorite ions.

9. A system for electrochemical reduction of a carbon source comprising:
a DC power supply; and
a reactor including an electrode that includes a catalytic component comprising a catalyst comprising Fe (iron) at 2-15 weight %, and Ti (titanium) at 0.3-5 weight %, Ni (nickel), and Zn (zinc) disposed in an aluminosilicate in electronic communication with the DC power supply.

10. The system of claim 9, wherein the reactor is a batch reactor or a continuous membrane reactor.

11. The system claim 9, wherein the batch reactor includes a jacket.

12. The system of claim 11, wherein the jacket configured to maintain a temperature from 60-70° C.

13. The system of claim 9, wherein the reactor is connected to a water spray jet.

14. The system claim 9, wherein an upper part of the batch reactor is configured to collect one or more product gas mixtures, and wherein the batch reactor defines a gas outlet.

15. The system of claim 9, wherein the electrode includes an anode and a cathode, and wherein the anode and the cathode are connected to each other by the catalytic component.

16. The system of claim 9, wherein the electrode includes a graphite semi-cone piece and insulated rods.

17. The system of claim 9, wherein a plurality of the graphite semi-cone piece are connected in series.

18. The system of claim 9, wherein the catalytic component is an anode and/or a cathode in an electrochemical cell.

19. The system of claim 9, wherein the catalytic component is a cathode and a carbon material is an anode of an electrochemical cell.
